(19) United States
(12) Patent Application Publication
(10) Pub. No.: US 2025/0134715 A1
(43) Pub. Date: May 1, 2025

(54) METHODS FOR SELECTIVE ABERRATION CORRECTION IN CORNEAL LASER ABLATION

(71) Applicant: Apollo Medical, LLC, San Diego, CA (US)

(72) Inventor: Manoj Motwani, San Diego, CA (US)

(21) Appl. No.: 18/763,706

(22) Filed: Jul. 3, 2024

Related U.S. Application Data
(60) Provisional application No. 63/524,813, filed on Jul. 3, 2023.

(51) Int. Cl. A61F 9/008; A61B 3/103

(52) U.S. Cl. CPC A61F 9/00814; A61B 3/1035; A61F 2009/00872; A61F 2009/0088; A61F 2009/00882

(57) ABSTRACT

Methods and systems for planning and performing a corneal laser ablation procedure that may comprise a wavefront aberrometer and a corneal topography measuring device. Data from each of these devices may be compared for generating an astigmatism treatment to be incorporated into a laser ablation treatment map. Corrections derived from epithelial thickness measurements may also be incorporated into the treatment map.

### CROSS REFERENCE TO RELATED APPLICATIONS

This application claims priority to U.S. Provisional Application 63/524,813, filed on Jul. 3, 2023.

### BACKGROUND

Selective laser ablation of the cornea has become a popular procedure to correct vision problems caused by various refractive anomalies of the eyes of a given subject.

As initial background information regarding the physiology of the eye, three fundamental components of the human eye as shown in FIG. 1 are the cornea 12, the lens 14, and the retina 16. The optical axis 18 of the eye is an imaginary line passing approximately through the peak of the cornea and normal to the corneal surface 12 straight back to a point on the retina 16. Ideally, light rays within a few millimeters of and parallel to the optical axis 18 that are incident to an eye will enter the cornea 12, pass through the lens 14 and will be focused essentially on a single point on the retina 16. The refractive power of the eye is typically measured in diopters and is the reciprocal of the focal length of the lens system formed by the cornea 12 and lens 14. Ideally, the power of the eye is such that the focal length is close to the axial length 20 of the eye. Typically, human eyes have a power of approximately 60 diopters, with about 40 diopters in the cornea and about 20 diopters in the lens.

Referring now to FIG. 2, for explanatory purposes, the cornea may be divided into an anterior portion 12a and a posterior portion 12b divided in FIG. 2 at the imaginary dotted line 12c running approximately through the middle of the stroma 34. The stroma 34 is the largest component of the cornea. The stroma 34 is a layered cellular structure that may be about 450-500 micrometers thick for a normal cornea and comprise around 90% of the total corneal thickness. The most anterior component of the anterior portion 12a of the cornea illustrated in FIG. 2 is the epithelium 32, which comprises several layers of cells that total typically about 50-60 micrometers thickness and are about 10% of the total corneal thickness. Although not shown in FIG. 2, there is a thin layer of water called the tear film that coats and lubricates the outer air exposed surface of the epithelium 32. The other layers of the cornea denoted 33, 35, and 36 in FIG. 3 are quite thin relative to the stroma 34 and the epithelium 32 although they can be significant functionally and clinically. The most posterior component of the posterior portion 12b of the cornea is the endothelium 36, which is a cell monolayer a few micrometers thick. The cornea further comprises thin acellular layers 33 and 35 at the boundaries between the stroma cells and the epithelium 32 and the endothelium 36 respectively, typically comprising collagen as well as other biological molecules. Although illustrated as single membranes 33 and 35 in FIG. 2, these layers have recognized sub-layers with different components and characteristics. The layer 33 between the epithelium and the stroma may comprise the epithelium basement membrane and Bowman's membrane. The layer 35 between the endothelium and the stroma may comprise Dua's layer and Descemet's membrane.

Common refractive errors of the eye include myopia, hyperopia, and astigmatism. With myopia, the anterior corneal surface curvature may be substantially spherical, but the curvature and hence the power is too large. With hyperopia, the anterior corneal surface curvature may also be substantially spherical, but the curvature and hence the power is too small. With astigmatism (also referred to as cylinder), the curvature of the anterior corneal surface and/or the lens 14 may have a non-spherical football type contour, with a different radius of curvature in different directions from the peak. For example, the anterior corneal surface could have a maximum power along a first circumferential meridian and a minimum power in a circumferential meridian approximately orthogonal to that. Although there are different ways to numerically represent it, a characterization of an astigmatic eye may include two values, one related to the maximum and minimum refractive powers in the different directions (e.g., the difference between these two powers) and an angle defining the deviation from horizontal of the lower power meridian.

Referring now to FIG. 3, refractive aberrations of an eye may be characterized by the coefficients of polynomials called Zernike polynomials (although other representations such as Fourier series can also be used). Myopia, hyperopia, and lower order astigmatism are characterized by coefficients of second order Zernike polynomials denoted 24 in FIG. 3. These are lower order aberrations (LOA). Optical aberrations of the eye can also include aberrations characterized by coefficients of third and higher order Zernike polynomials and may be referred to as higher order aberrations (HOA) denoted as 26 in FIG. 3. High order aberrations characterized by Zernike polynomials include aberrations known as coma, trefoil, quadrafoil, and spherical aberration. As can be seen in FIG. 3, there is also higher order astigmatism known as “secondary” astigmatism that may be present, but when the term astigmatism is used alone, it is lower order astigmatism that is being referred to. The refractive properties of any given eye as a whole may be expressed as a combination of one or more of these Zernicke characterized refractive aberrations in varying amounts. Also, the surface contour of the anterior portion of the eye can also be expressed as a combination of one or more of these Zernicke polynomials.

The laser ablation procedures that have been developed have attempted to correct untreated refractive aberrations of the eye by altering the anterior shape of the cornea to remove or otherwise counteract the refractive aberrations of the untreated eye. Fundamentally, portions of the cornea are ablated away to change the shape of the air/cornea interface in a manner that reverses the refractive aberrations of the untreated eye.

Laser ablation procedures perform ablation of stromal tissue 34 and come in two basic types that differ in how the stroma is exposed to the laser for the ablation procedure itself. In photorefractive keratectomy (PRK), the epithelium 32 over the portion of the stroma 34 to be treated is removed to expose the stromal tissue for laser ablation. This removal may be done mechanically with a brush for example, chemically, with laser pulses, or possibly a combination of such techniques. The underlying stromal tissue is then ablated to change the shape of the cornea as desired. After the procedure, the epithelium grows back over the stromal tissue in a few days or weeks after the surgery. With laser assisted in-situ keratomileusis (LASIK), a flap is created which may be about 100 micrometers deep through the epithelium and the outermost portion of stromal tissue with a mechanical blade or a laser. This flap is pulled back, exposing anterior stromal tissue which is then ablated to change the shape of the cornea as desired. After the stromal ablation, the flap is folded back over the surgical site where it heals back in place. Typically, LASIK has a shorter recovery period and less post-surgical discomfort than PRK. In both cases, stromal tissue is selectively removed by laser ablation to correct refractive errors. All the methods and apparatus described herein are equally applicable to either or both PRK, LASIK, or other to be developed surgical techniques involving modifying stromal tissue to improve vision.

In some versions of these procedures, such as shown in FIG. 4, an examination of a patient with, for example, a phoropter 44 may be performed to generate a “manifest” refraction measurement to generate a correction prescription 46. With a phoropter 44, by looking through a sequence of different lenses and determining which corrections look best to the patient in terms of perceived visual quality, sphere and cylinder corrections are found that compensate for the patient's myopia, hyperopia, and/or astigmatism. Instead of being used to generate a prescription for glasses or contact lenses, the manifest refraction prescription 46 may be used to generate a treatment map 48a defining how to re-shape the cornea 12 to generate the phoropter determined sphere and cylinder corrections. The treatment map 48a defines stromal tissue removal depths at different locations on the anterior corneal surface to generate a desired cornea curvature after the laser ablation surgical procedure is complete.

As an alternative and/or supplement to the manifest refractive measurements of FIG. 4, more automated and objective methods and systems have been developed to characterize the refractive properties and shape characteristics of the eye and generate treatment maps for use in anterior corneal laser ablation procedures. For example, a technique known as wavefront guided laser surgery has been developed. Referring to FIG. 5, an instrument called a wavefront aberrometer 52 is used to characterize the properties of the patient's eye. These instruments, such as a Hartmann-Shack wavefront aberrometer, may shine a spot of light onto the retina, and the retina reflected wavefront that exits the eye is detected through a lens array. As the reflected light travels out of the eye, the wavefront exiting through the pupil opening is distorted, and detecting these distortions can characterize the total optical aberrations 53 introduced into the light beams by the optical elements and interfaces of the patient's eye. From this information, a treatment map 48b may be generated. In some cases where wavefront aberrometry is performed, manifest refraction measurements 54 made with a phoropter are used for some of the corrections incorporated into the treatment map 48b, while in other cases, the manifest refraction is not utilized at all.

A deficiency with wavefront aberrometry is that it is difficult to effectively measure and correct HOAs. Typically, HOAs are most pronounced in subjects who have damaged, diseased, or otherwise abnormal corneas. Although wavefront measures the entire ocular focusing system, including HOAs, a modality was introduced in the field of laser eye surgery known as topographic guided ablation, which provides more enhanced imagery of the anterior corneal surface thereby providing the ability to more effectively map and define HOAs as irregularities in the surface elevation of the anterior corneal surface. Although topographic guided ablation was initially used for repair of damaged, diseased, or otherwise abnormal corneas, it is also used for primary refractive correction on patients with otherwise normal corneas.

Referring now to FIG. 6, for topographic guided ablation, rather than using a wavefront aberrometer 52, a corneal topology measuring instrument 56 is utilized. Corneal topology measuring instruments 56 are configured to generate high resolution elevation measurements 55 of the anterior surface of the cornea. Such maps can be very high resolution and may map the elevation of many thousands of points in a corneal area of a few millimeters diameter. These devices detect light reflected from the tear film, anterior epithelium surface, and/or epithelium basement membrane. The WaveLight Topolyzer VARIO from Alcon is one example of a commercially available corneal topology measuring instrument 56 that uses a Placido ring illumination and analysis technique. Another method of acquiring corneal topology is known as a Scheimpflug imaging system. This technique acquires images of slices of the eye at different angles. A Scheimpflug camera may acquire a variety of structural information regarding the anterior and posterior surfaces of the cornea with these slice images. This information may include high resolution elevation data for the anterior corneal surface. From this information, a treatment map 48c may be generated. In some cases where anterior topography is performed, manifest refraction measurements 54 made with a phoropter are used for some of the corrections incorporated into the treatment map 48b, typically the sphere or defocus correction.

A fundamental difference between wavefront aberrometry and corneal topography is that wavefront aberrometry provides information on the combined optical properties of the eye as a whole (e.g. including the anterior epithelium/corneal region, the posterior occular region, the lens, the retina, etc.), whereas corneal topography provides information only regarding the surface shape of the anterior surface of the eye without measuring the optical characteristics of other optical elements of the eye. Therefore, since established science has always held that astigmatism can be significantly affected by posterior ocular irregularities, topographic measurements were not used to correct astigmatism until the results of the Inventor's clinical studies were completed in 2016 that challenged this established but incorrect idea. Instead, topographic-guided ablation systems relied on manifest refraction measurements for input to treat astigmatism, which is an essentially lower order aberration. Since topographic measurements of lower order astigmatism often differed from manifest measurements, established science theorized that the differences were due primarily to posterior ocular irregularities; therefore, requiring the use of manifest measurements for correction of astigmatism whenever topographic measurements differed from manifest.

Topographic-guided ablation systems have now been significantly improved based on the new science established from the Inventor's studies, which provided new systems and methodologies for the use of topographic measurements to not only ablate HOAs, but to reduce astigmatism in accordance with anterior corneal astigmatism based at least in part on topographic measurements.

As shown in FIG. 7, the treatment map 48a, b, c, however it is generated, may be used as an input to a laser system 62 that performs the laser ablation accordingly. First, the treatment map 48a, b, c is used to generate a laser ablation pattern 64 which defines laser intensity, pulse duration, number of pulses, and other laser beam parameters necessary to accomplish the depths of corneal removal specified in the treatment map 48a, b, c. This laser ablation pattern 64 may also take into account such positional factors as the slope of the cornea at locations away from the apex. Because the laser ablation pattern 54 will need to take into account specific characteristics of the laser 68 being used in the system 62, software that generates the laser ablation pattern 64 from the treatment map 48a, b, c is typically closely associated with the laser system 62 itself. The laser ablation pattern 64 is used by a laser control system 66 to control laser position, pulse duration, and the like for the laser 68, which may, for example, be an excimer laser, to emit the appropriate laser pulses to perform the ablation surgery.

Since PRK and LASIK were introduced, the aim has been to achieve the anterior corneal shape from an ablation pattern derived from devices and methods that have an established medical and scientific basis for providing the best possible optical quality. Although this is the aim, the systems, methods and reasons to achieve such anterior corneal shape vary and those variances relate as much to differing scientific views as to available technology. A large amount of research has been performed by surgeons in the field to attempt to define the best way to treat various refractive conditions of the eye to produce the best outcomes for subjects of these procedures. Such research continues to be performed as available measurement technologies have become available.

Commercialized corneal laser ablation systems and methods have been based on the premise that the ablation pattern configured for a subject, such as for astigmatism, should be derived from a quantification of optical quality that is determined using subjective and/or objective data derived from “whole” eye measurements, including anterior and posterior regions. The reasoning for this premise is based on established medical and scientific postulation that astigmatism is caused by refractive anomalies that are present throughout a subject's entire ocular focusing system, including the posterior ocular region. Under this premise, in order to achieve the best possible optical quality (based on established medical and scientific principles) an ablation pattern for correcting astigmatism had been and was largely medically and scientifically required to be derived from “whole” eye measurements prior to the Inventor's studies in 2016.

The Inventor has challenged such established medical and scientific principles and has developed systems and methods for corneal laser ablation that are novel and contrary to certain established medical and scientific principles. It was the Inventor's premise that the ablation pattern for a subject with astigmatism should not be derived solely from manifest measurements, but rather from a quantification of anterior surface features which are necessary to create a more uniform cornea to make the stromal surface as smooth and spherical as possible.

Results from the Inventor's clinical studies found that the primary reason for the differences between topographic measurements and manifest measurements was not posterior ocular irregularities as postulated by medical and scientific theory, but rather the effects of anterior corneal HOAs on such measurements. The clinical studies showed that if HOAs of a subject were not first ablated in accordance with topographic measurements, manifest measurements of that subject generally showed false astigmatism measurements due to the masking effect of the HOAs. The more significant the anterior corneal HOAs, generally the more significant the false astigmatism measurements. As a result of the studies, the Inventor created the LYRA™ (i.e. Layer Yoked Reduction of Astigmatism) protocol, which is a medical methodology developed by the Inventor which used corneal astigmatism based on topographical measurements, as a better methodology for programming of ablation patterns to correct astigmatism. Systems and methods such as those described in the Inventor's U.S. Pat. No. 10,857,032 (incorporated herein by reference in its entirety) are modalities that utilize these principles.

Another significant variable in the ocular focusing system is the epithelial layer of the cornea, which is the outermost or most anterior layer. This epithelial layer is the only corneal layer that regenerates; the underlying stromal layer (the “meat” of the cornea where laser ablation is performed) and the endothelium (the innermost layer which pumps fluid out of the cornea to keep it optically clear) do not regenerate. But, since the epithelial layer does regenerate, such layer can significantly vary based on a number of factors, including HOAs, disease or trauma. However, although the epithelium may vary, it is not considered to be variable in the normal Gullstrand models of the eye, which are used for existing topographic or wavefront mapping. As a result, existing modalities fail to properly measure the epithelial thickness over the anterior cornea following the shape of the cornea underneath. The Inventor's studies have shown that the epithelium varies in thickness rather dramatically. It thickens to fill in flatter “low” stromal areas and thins over steeper “high” stromal areas (referred to as “epithelial compensation”). This has the effect of masking some of the anterior stromal corneal irregularities, and thus creating topography and wavefront mapping that would not properly measure the actual corneal irregularity.

Over the decade prior to 2017 some experimental devices were used by surgeons to measure epithelial thickness and document epithelial compensation. Furthermore, attempts have been made to map the epithelial basement membrane, which would map the anterior epithelium-stromal border directly. In late 2016 and 2017 the Inventor realized that the epithelium had to be playing a part in compensation for corneal irregularities as he noticed evidence of change on topographic maps in the days and weeks after laser treatment with the LYRA™ Protocol. In 2017, Optovue introduced the first commercial epithelial thickness mapping devices and in September 2017 the Inventor obtained one for use and began correlating epithelial compensation with anterior corneal irregularities. He published this information in a paper in 2020 describing reasons for inaccuracy of outcomes after treatment with Contoura with LYRA™ Protocol for primary LASIK corrections, affecting a percentage of primary LASIK corrections. This led the inventor to create a system, methodology and device to measure and treat epithelial compensation of corneal irregularity along with topographic guided ablation to make a more uniform cornea. Systems and methods such as those described in the Inventor's U.S. Pat. No. 10,857,033 (incorporated herein by reference in its entirety) are modalities that utilize these principles.

Further research led to the discovery of high levels of epithelial compensation, with 30 plus microns of epithelial compensation in certain situations; thereby significantly masking stromal irregularity. The Inventor then developed a new medical procedure based on his clinical studies involving epithelial compensation, the Corneal Repair Epithelium Adjusted Topography Enhanced Protocol (CREATE™ Protocol) to address this medical condition. This protocol treats the stroma masked by the epithelial compensation and then utilizes topographic guided ablation using the LYRA™ Protocol to increase higher order aberration reduction and create as uniform, regular cornea as possible.

Using the CREATE protocol, the Inventor began to treat this masking by performing surface excimer laser ablation (phototherapeutic keratectomy or PTK) to the central anterior epithelium to the depth determined by the thickest area in the central 5 mm optical zone. As epithelial tissue and stromal tissue are removed similarly by excimer laser, this effectively removed anterior stromal irregularity that could not be measured otherwise due to the epithelium masking. This was followed by topographic guided ablation that removed the remainder of irregularity measurable by topography.

The results from the Inventor's clinical studies using the new CREATE™ Protocol in a newly configured methodology which included epithelial compensation factors on subjects diagnosed with keratoconous, an eye condition in which the cornea gets thinner and gradually bulges outward into a cone shape, showed a typical HOA reduction of 57% (in contrast with prior methods getting only about 31% reduction), and a dramatic decrease in a subject's optical aberration. HOA reduction may have improved even more with the CREATE™ Protocol if the Inventor didn't limit the corrections performed to the astigmatism correction limitation of the Contoura laser system. The corresponding increase in vision significantly improves the outcomes for treatment of keratoconus over prior procedures. The beneficial results from this breakthrough clinical procedure for keratoconus by the Inventor have been submitted for publication to the medical community and were an inspiration for the Inventor in creating the novel systems and methodologies embodied herein.

Another technique that has been experimented with over the past decade is a system referred to as ray tracing. Ray tracing is a computer-based method used to calculate an ablation profile for a refractive laser by incorporating data derived from several types of measurements. It may incorporate a combination of information such as wavefront guidance to determine theoretical posterior ocular aberrations as well as axial length of the cornea and topography information to determine the curvature of the cornea at each point measured, and therefore the corneal power and astigmatism as well. It attempts to treat all of these per point by creating a ray tracing/vector diagram for each point and therefore treat, theoretically, all sources of aberrations through the entire ocular focusing system. Although theoretically perceived to be a potentially more effective corneal laser ablation system, it still has not been commercially perfected and, as will be described later herein, has existing deficiencies, some of which may be overcome by embodiments provided in this invention.

It should be noted that this Background is not intended to be an aid in determining the scope of the claimed subject matter nor be viewed as limiting the claimed subject matter to implementations that solve any or all the disadvantages or problems presented above. The discussion of any technology, documents, or references in this Background section should not be interpreted as an admission that the material described is prior art to any of the subject matter claimed herein.

### SUMMARY

Embodiments of this invention include corneal laser ablation methods and systems configured and programmed to perform the methodologies derived from the disclosures herein. The summary below is directed to some of the disclosed methods of the disclosure herein, not to all of the inventive embodiments described herein.

In one embodiment, a method of performing a corneal laser ablation procedure on an eye of a subject, the method comprises generating a treatment map. The generating may comprise acquiring measurements of one or more lower order whole eye optical aberration characteristics at least in part via retinal reflection measurements and deriving therefrom a first astigmatism characteristic representative of a lower order whole eye astigmatism. The method may further comprise acquiring measurements of one or more lower order elevation characteristics of the anterior corneal surface of the eye at least in part from one or more of either tear film, anterior epithelium, and/or epithelium basement membrane reflection measurements and deriving therefrom a second astigmatism characteristic representative of a lower order anterior corneal surface astigmatism. The first astigmatism characteristic may be compared with the second astigmatism characteristic to generate an estimate of an amount of difference between lower order whole eye astigmatism and lower order anterior corneal surface astigmatism. Based at least in part on the comparing, a third astigmatism characteristic is determined for inclusion in the generation of the treatment map. The third astigmatism characteristic is determined using either (1) one or both of the first astigmatism characteristic and the second astigmatism characteristic or (2) a derivative of one or both of the first astigmatism characteristic and the second astigmatism characteristic. The method may further comprise generating the treatment map using at least in part the third astigmatism characteristic, creating an ablation pattern that can be used by a corneal laser ablation device from the treatment map, and using a corneal laser ablation device to apply laser pulses to the eye in accordance with the ablation pattern.

In some embodiments of the above method, the estimate may be compared to a threshold. In some embodiments, the threshold may be user-settable.

In another embodiment, a method of generating a treatment map in order to create an ablation pattern that can be used for performing a corneal laser ablation procedure on an eye comprises measuring a dataset of lower and higher order whole eye optical aberration data, measuring a corresponding dataset of lower and higher order anterior corneal surface aberration data, and comparing the dataset of anterior corneal surface aberration data with the corresponding dataset of whole eye optical aberration data. Based at least in part on the comparing, one or more of the following acts (a) through (f) may be performed: (a) selecting a subset of the measured corneal surface aberration dataset for a corresponding subset of lower order and/or higher order aberration treatment, (b) selecting a subset of the measured whole eye optical aberration dataset for a corresponding subset of lower order and/or higher order aberration treatment, (c) selecting a derivative determined at least in part from both the measured whole eye optical aberration dataset and the measured corneal surface aberration dataset for a subset of lower order and/or higher order aberration treatment, (d) selecting a derivative determined by an output of a computation that uses a first percentage of a subset of the measured corneal surface aberration dataset and a second percentage of a corresponding subset of the measured whole eye optical aberration dataset as inputs for a subset of lower order and/or higher order aberration treatment, (e) selecting a derivative determined from an algorithm using the selected subset of corneal surface aberration data and a corresponding selected subset of whole eye optical aberration data as inputs for a subset of lower order and/or higher order aberration treatment, (f) selecting a derivative determined from an algorithm using the difference in the selected measured subset of whole eye optical aberration data and the selected measured subset of corneal surface aberration data as inputs for a subset of lower order and/or higher order aberration treatment. One or more of the chosen options may be used to define a treatment set of lower order and/or higher order aberrations for correction and the treatment set of lower order and/or higher order aberrations may be combined for correction to generate the treatment map.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1 is a cross section of eye anatomy.

FIG. 2 is a close-up view of the cornea of FIG. 1.

FIG. 3 illustrates low order and high order aberrations.

FIG. 4 is a prior art method for treatment map generation using a phoropter.

FIG. 5 is a prior art method for treatment map generation using a wavefront aberrometer.

FIG. 6 is a prior art method for treatment map generation using a corneal topography measuring instrument.

FIG. 7 illustrates using a treatment map in a corneal laser ablation system.

FIG. 8 is a treatment map generation system in accordance with some embodiments of the invention.

FIGS. 9A, 9B, and 9C illustrate a hardware configuration that may be suitable for implementing the systems in accordance with FIG. 8.

FIG. 9D illustrates another hardware configuration that may be suitable for implementing the systems in accordance with the principles of FIG. 8.

FIG. 10A is an example user-interface that may be associated with the system of FIG. 8.

FIG. 10B is another example user-interface that may be associated with the system of FIG. 8.

FIG. 11 is a flowchart of a method of corneal laser ablation in accordance with some embodiments of the invention.

FIG. 12 is a flowchart of another method of corneal laser ablation in accordance with some embodiments of the invention.

FIG. 13 is a flowchart of another method of corneal laser ablation in accordance with some embodiments of the invention.

FIG. 14 illustrates using a treatment map in a method of making lenses for glasses, contact lenses, and/or lenses for cataract surgery.

FIG. 15 is a flowchart of another method of corneal laser ablation in accordance with some embodiments of the invention.

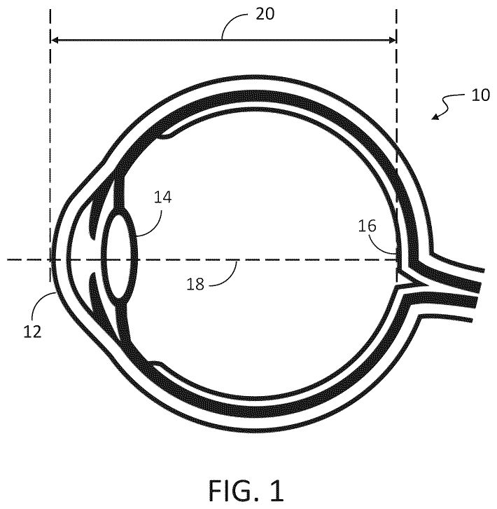

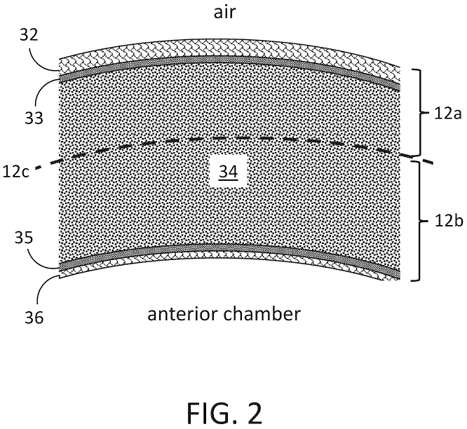

### DETAILED DESCRIPTION

The following description and examples illustrate e some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

To facilitate an understanding of the various embodiments described herein, a number of terms are defined below.

PRK: An acronym for Photorefractive Keratectomy. PRK is a laser ablation procedure wherein the epithelium covering the corneal stromal tissue is removed mechanically, chemically, and/or with laser ablation as part of a laser ablation refractive correction treatment on an eye. Following PRK, the epithelium grows back over the corneal stromal tissue that has been modified with laser ablation for refractive corrections.

LASIK: An acronym for Laser-Assisted In Situ Keratomileusis. LASIK is a laser ablation procedure wherein a hinged circular or approximately circular flap of typically about 100 to 160 micrometer thickness and 8 to 10 mm diameter is pulled off of the anterior portion of the cornea to expose a surface of corneal stromal tissue to which laser ablation for refractive correction is applied. The perimeter of the flap may be cut mechanically with a blade or may be cut with a laser. The flap thickness typically spans the epithelium, the basement membrane, and an anterior portion of the corneal stromal tissue. After the laser ablation for refractive correction is performed, the hinged flap is placed back over the treated corneal tissue. With LASIK, the epithelium remains substantially intact, and the post treatment healing occurs around the perimeter of the flap and at the corneal tissue interface underneath the flap.

Anterior Ocular Region: The anterior cornea that includes the tear film, the epithelium, the basement membrane, and corneal stroma.

Posterior Ocular Region: All structures of the eye beneath (posterior to) the anterior ocular region. These include, for example, the posterior surface of the cornea and the lens.

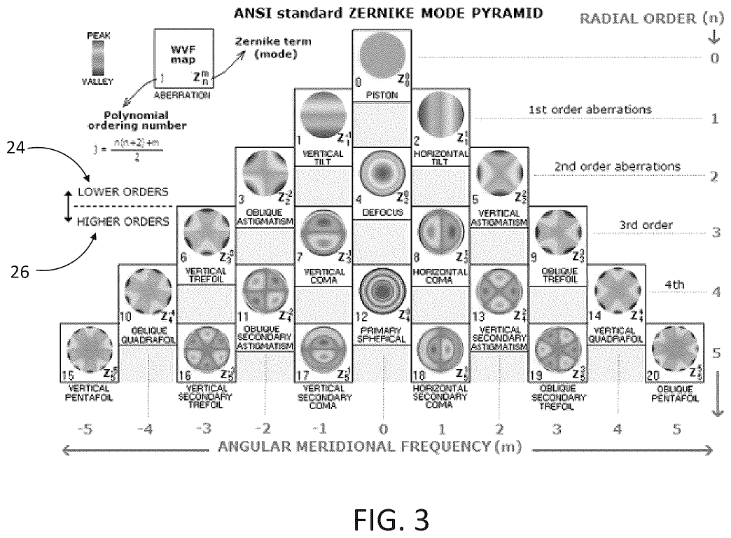

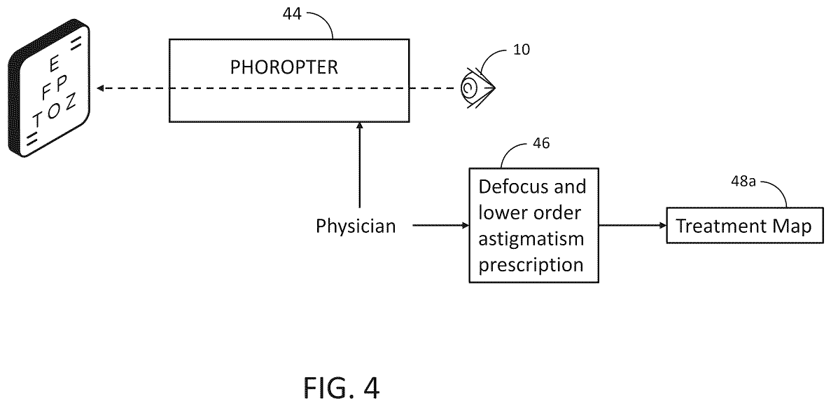

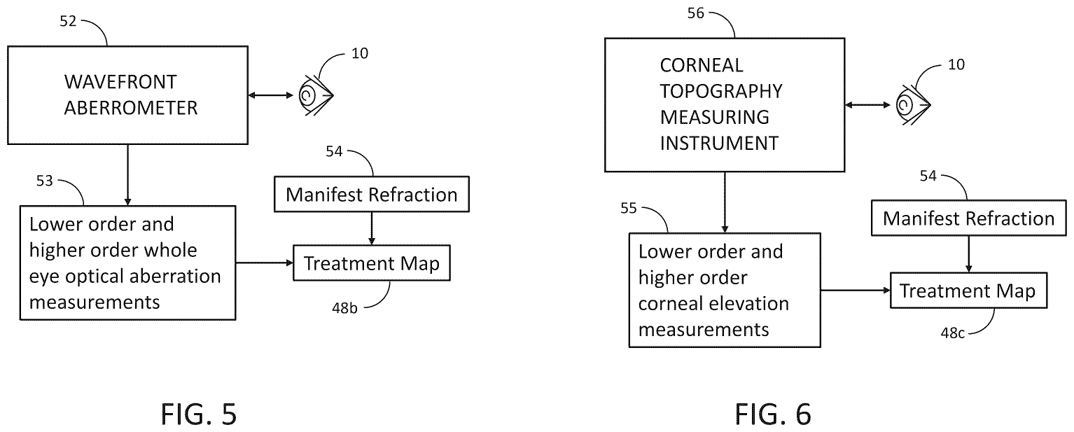

Astigmatism: A shape characteristic and/or optical refractive characteristic of one or more surfaces of an optical system that correspond to the Zernicke polynomials of radial order 2 and angular order +2 and −2 (see FIG. 3), usually characterized by a surface shape with different curvature in different directions over the surface. An optical system may have multiple refractive surfaces each of which has an individual astigmatism shape characteristic. The astigmatic shape characteristics of the individual surfaces will combine to generate a total optical refractive astigmatism characteristic of the optical system as a whole. The contribution that each refractive surface makes to the total astigmatism of the optical system will depend on the astigmatic shape characteristic of the surface and the index of refraction change occurring across that surface. For an eye, surfaces along the optical path of the eye that may have an astigmatism shape characteristic include the anterior corneal surface, the posterior corneal surface, the lens, and the retina. The term astigmatism characteristic or astigmatic characteristic may be used herein to refer to a topographical shape of a single surface or to an optical refraction characteristic of either a single surface or a collection of surfaces forming all or part of an optical system. Astigmatism measured by a topographic measuring technology refers to an astigmatic physical contour shape characteristic of the surface measured. Astigmatism measured by wavefront aberrometry refers to the astigmatic optical refractive aberration generated as the wavefront travels through the one or more surfaces during the measurement. As used herein, refractive astigmatism refers to an astigmatic optical refractive property of a surface or collection of surfaces and topographic astigmatism or shape astigmatism refers to an astigmatic physical contour of a single surface of an optical system. These are not necessarily identical but are related because refractive astigmatism of an optical system or part thereof is produced by topographic astigmatism of the surface or surfaces making up the optical system. An astigmatism characteristic, whether topographic or refractive, can be characterized in different ways. For example, an astigmatism characteristic of a surface or system may be expressed as a degree of astigmatism present in a surface or system, or it may be expressed as a required change or correction to a surface or system in order to make that surface or system devoid of topographic or refractive astigmatism. In some cases, the term astigmatism as used herein is explicitly applied to the topographic shape of a surface. In some cases, the term astigmatism as used herein is explicitly applied to the refractive properties of all or part of the eye. In some cases, the term astigmatism is used alone in which case it may refer to either or both depending on the context.

Posterior Ocular Astigmatism: Astigmatic refractive aberrations caused by astigmatic shape characteristics of the eye in the posterior ocular region.

Anterior Corneal Astigmatism: Astigmatic topographic shape characteristics of the anterior surface of the cornea.

Posterior Corneal Astigmatism: Astigmatic topographic shape characteristics of the posterior surface of the cornea.

Lenticular Astigmatism: Astigmatic refractive aberrations of the lens as a whole.

Retinal Astigmatism: Astigmatic topographic shape characteristics of the retina.

Whole Eye Astigmatism: Astigmatic refractive aberrations caused by the combination of astigmatic shape characteristics present throughout the entire eye focusing system.

Manifest Astigmatism: The astigmatic refractive aberrations perceived by a subject. The manifest astigmatism may or may not be the same as the whole eye astigmatism because the visual perception of the subject may be further influenced by shape and or refractive higher order aberrations also present in some or all of the eye.

Epithelial Compensation: Epithelial growth that is reduced or increased due to aberrations in the anterior ocular region that results in differences in epithelial thickness over a defined area of the anterior ocular region. Numerically, epithelial compensation can be quantified as the thickness difference between the thickest part of the epithelium and the thinnest part of the epithelium in a defined region of the anterior surface of the eye, for example in a region of defined diameter centered on the corneal apex.

Lower Order Aberrations (LOA): Shape and/or refractive characteristics of the eye or one or more parts of the eye that correspond to Zernicke polynomials of radial order 2 or less. As described above with respect to the term astigmatism, lower order aberrations may, depending on the context, refer to the topographic shape characteristics of an individual refractive surface or may refer to the optical refractive aberrations of an optical system or portion thereof with more than one refractive surface.

Higher Order Aberrations (HOA): Shape and/or refractive characteristics of the eye or one or more parts of the eye that correspond to Zernicke polynomials of radial order 3 or more. As described above with respect to the term astigmatism, higher order aberrations may, depending on the context, refer to the topographic shape characteristics of an individual refractive surface or may refer to the optical refractive aberrations of an optical system or portion thereof with more than one refractive surface.

Based on clinical studies performed by the Inventor over many years, the Inventor had previously concluded that in order to achieve the best optical quality for most subjects, a corneal laser ablation system should be configured with the particular devices and data input necessary to derive a laser ablation pattern calculated to create the most uniform cornea possible. The systems and methods that can be created from the information and embodiments herein, further expand on the Inventor's challenge of established medical and scientific principles that it is the uniformity of the cornea, not the subjective and/or objective data of whole eye measurements, that is the key to creating the best possible optical quality for a subject. Systems and methods described herein expand the applicability of the treatments developed by the Inventor to additional patients and provide methods and systems for corneal laser ablation treatments that are more easily used and flexible for surgeons to adapt to the wide variety of patients presented to them for treatment.

Elements of a basic novel system in accordance with embodiments described herein may include:

Topography imaging (e.g. Placido or Scheimpflug imaging) that uses a best fit sphere or other software technology to image elevations and depressions in the anterior corneal surface to create a map of the lower order and higher order aberrations of the anterior corneal surface. This will provide the lower order aberrations in the form of topographic astigmatism of the anterior cornea in (for example) magnitude of diopters and the axis of the topographic astigmatism once the higher order aberrations are removed.

Wavefront imaging utilizing an aberrometer such as Hartmann-Schack. Alternatively or in combination, an automated or manual phoropter may be used. This will provide whole eye higher order refractive aberrations as well as whole eye lower order refractive aberrations such as a magnitude and axis of refractive lower order astigmatism. The wavefront aberration map will include all sources of refractive lower order astigmatism including refractive posterior ocular astigmatism that can be caused from posterior cornea, crystalline lens, the retina, or any other source of refractive posterior ocular astigmatism. This data may be important to be examined as it contains one of the two variable HOA/LOA sources in the eye, the crystalline lens.

Epitheilal compensation data of anterior stromal irregularity cannot be measured by the above two sources and constitutes the second variable HOA source in the eye. Epithelial compensation occurs when the epithelium thins over stromal irregularity elevations and thickens over stromal irregularity depressions. This can be measured either by OCT mapping of epithelial thickness, or by technology mapping the edge of the stroma/basement membrane. This interface can be mapped with certain technologies as well. This epithelial compensation map can be matched to the anterior corneal elevation map from topography.

Measurements from an interferometer can provide axial length which can be used with the corneal topography information to provide lower order spherical power information.

A processor configured to use an algorithm to combine data from the wavefront aberrometer, the corneal topography measuring device, and/or the epithelial thickness measuring device into a treatment map for a corneal laser ablation treatment

A tracking system that uses a CCD camera to image corneal and iris features for each one of these ocular imaging technologies to align the maps appropriately.

A software system that uses comparative technology or artificial intelligence technology to align the imaging maps utilizing any or all of the following: the higher order aberration maps from topographic imaging and wavefront, the lower order aberration maps from the topography imaging and the wavefront imaging (and/or manifest), and the elevation/depression map from the anterior corneal imaging and the epithelial compensation.

A software rejection technology that rejects the combined map if too many disparities are present, or rejects the wavefront map if the anterior topography map and wavefront map show similar HOA and/or lower order astigmatism magnitude and axis, and if HOA maps demonstrate senile lens changes.

An axial length auto refraction technology that utilizes the wavefront imaging to provide a refraction of the eye that provides spherical power information.

A final software imaging map that incorporates any or all: HOA, LOA including astigmatism magnitude and sphere, spherical aberration and spherical power. This information can be transferred to an excimer laser system for laser correction of HOA and LOA to create an ocular system that minimizes HOA and LOA while eliminating variables from epithelial compensation or posterior ocular astigmatism sources.

The Inventor's findings concerning epithelial compensation of HOA/corneal irregularity has proved to be highly important for not only corneal laser repair of past surgery, disease, trauma, etc, but also for treating and/or preventing inaccurate outcomes of primary virgin eye LASIK and PRK procedures. As a result, the Inventor has configured new systems and methodologies using epithelial compensation principles along with the Inventor's LYRA™ and CREATE™ Protocols to significantly enhance corneal laser ablation and improve resulting outcomes. An advantageous feature of the embodiments of the new systems and methodologies is that it creates an ablation pattern that shapes the stromal surface that the epithelium grows over as smooth and spherical as possible, minimizing post-surgical epithelium issues. The configurations embody essentially three separate systems that can deliver data using a processing methodology that can be incorporated into a comprehensive laser ablation treatment map to allow for better corneal uniformity, elimination of most or all ocular focusing aberrations, and prevention of further epithelial compensation. An embodiment combines these three technologies, more fully described herein, to create a final HOA and LOA map for a particular eye.

In another embodiment the data in creating the final HOA and LOA map is subjected to comparative analysis to eliminate and/or require rescan of aberrant data that could include data from operator error.

Inventive embodiments described herein may utilize three different technologies to measure HOAs and LOAs of the cornea. The first technology is topography mapping, which can be from either Placido or Scheimpflug imaging. The second technology is wavefront imaging, which maps whole eye HOA and LOA and axial length, such as from Hartmann-Shack. The third technology is epithelial mapping measurement by either OCT or other technology. Certain embodiments herein include combining the data from these different imaging technologies to create a comprehensive map for treatment of all HOAs and LOAs, including the HOA masked by epithelial compensation. This system is designed to minimize all HOA and LOA in the ocular focusing system to create as close to a virtually perfect ocular focusing system as possible by treating the combined data on the cornea with an excimer laser system to make a more uniform cornea.

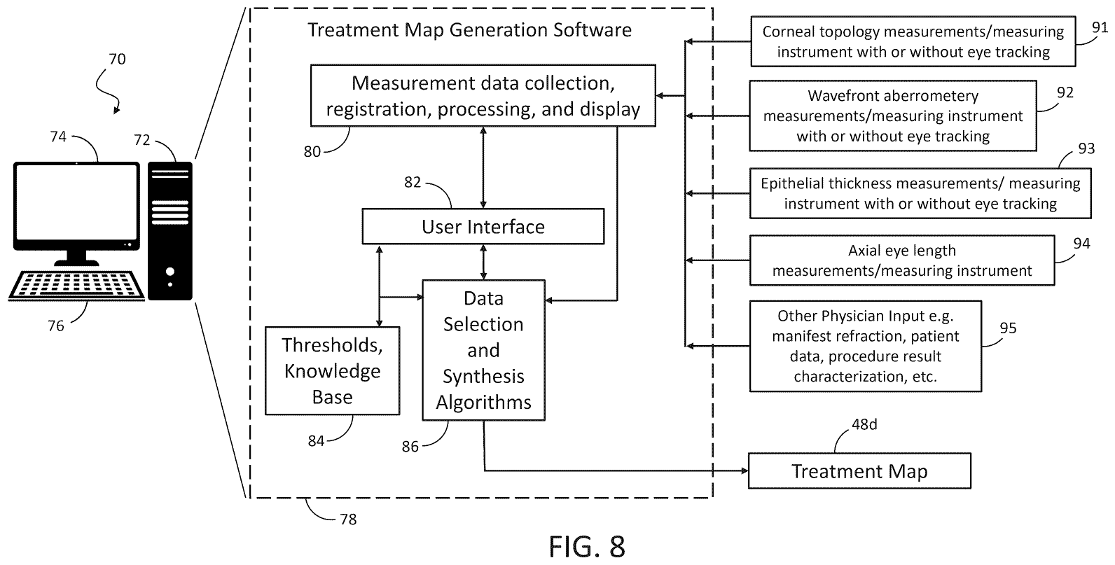

An example of a system for implementing the above principles is shown in FIG. 8. Referring now to this Figure, the system includes a general-purpose computer system 70 which hosts or executes, directly or indirectly, treatment map generation software 78 described in more detail below. The treatment map generation software 78 may reside on and be executed entirely by/on a local computer processing system 72 located at, for example, a LASIK surgeon's office or clinic. Alternatively, the treatment map generating software may be hosted on the Internet and accessed, for example, with a browser or other software that is executed on the processing system 72. The treatment map generating software may comprise processor executable instructions stored in a computer readable memory wherein the instructions are configured to cause a general purpose computer to perform the acts described herein for treatment map generation. As shown in FIG. 8, the processing system 72 may in some embodiments further be coupled to one or more I/O devices such as a keyboard and/or mouse 76 and a display 74.

The treatment map generation software 78 includes a measurement data collection, registration, processing, and display module 80 that receives clinical and physiological information from a variety of measurement modalities described further below. The treatment map generation software 78 further comprises data selection and synthesis algorithms 86 that uses the measurements data gathered and processed by the module 80 to generate an output treatment map 48d. In the embodiment of FIG. 8, which data is used and how that data is used by the data selection and synthesis algorithms 86 may be controlled and mediated through a user interface 82. In addition to allowing user selection of measurement modality data to use for the treatment map 48d, the user interface may also control the use of thresholds 84 and a knowledge base 88 to allow user-friendly and intelligent treatment map 48d generation that allows a wide variety of physician control and automated treatment map 48d generation.

The measurement data collection, registration, and processing module 80 can receive measurement data about a subject's eyes from one or more of a variety of sources. In FIG. 8, measurement inputs 91, 92, 93, 94, and 95 are illustrated as connecting to module 80. It will be appreciated that from a hardware perspective, the measurement inputs to module 80 will physically enter the system via the processing system 72 which will store them in a memory in the processing system 72 where they can be accessed by the treatment map generation software 78. The measurement data used by the module 80 may be in a variety of formats and may enter the system in a variety of ways. For some data in some embodiments, relevant data can be entered manually into the computer system 70. This would typically be applicable for clinical data such as manifest refraction measurements, patient information such as age, gender, other clinical information about the patient, follow up results, etc. This information is designated 95 in FIG. 8. Other information potentially received by the module 80 involves outputs of measurement devices. Some of this data could be entered manually as well but in many embodiments the processing system 72 would be directly connected with, connected over a network (including possibly the Internet or Intranet) or even incorporated into one or more of the measuring instruments 91, 92, 93, 94. Another possibility is that data generated by one or more of the measuring instruments could be stored on a flash drive or other memory technology and transferred from a measuring instrument to the processing system 72 manually.

Turning now to the measurement modalities themselves, the system of FIG. 8 may comprise a corneal topology measuring instrument 91 such as Placido or Scheimpflug imaging, and a wavefront aberrometer 92 such as Hartmann-Schack, and an epithelial thickness measuring instrument 93, such as optical coherence tomography (OCT). Although OCT devices have been commercialized and made available, its use has been limited because of the lack of understanding of how the epithelium affects corneal laser ablation procedures and the lack of recognized medical protocols in that regard. The Inventor has performed clinical studies to better understand the effects of epithelial compensation on the anterior cornea surface and has developed the novel medical protocol, the CREATE™ Protocol to, among other things, assimilate epithelium mapping and OCT devices into new systems and methods for corneal laser ablation.

These three instruments 91, 92, and 93 may or may not include eye tracking functionality such as a optical imaging camera configured to establish eye markers for tracking and alignment of the maps generated by each of the devices. When included, such eye tracking functionality can help ensure proper registration of the images and measurements made by the devices so the outputs can be overlaid and appropriately compared and processed.

Other data gathering instruments that may be a part of the system include an axial eye length measuring instrument 94 which may be dedicated measurement hardware such as an interferometer but which may also be accomplished with OCT or other technologies. As noted above with respect to inputs 95, phoropter measurements may also be gathered to obtain manifest defocus and manifest astigmatism values for entry into the system. Although manifest refraction data would not necessarily be required when a wavefront aberrometer 92 is provided, it can still be useful for comparison with the objective measurements from the other instruments and in some cases a surgeon may want to perform laser ablation based on correcting the manifest refractive error.

As noted above, data from these instruments is collected, registered, and processed by treatment map generation software 78. The registration and selection enables the epithelial thickness measurement and corneal topology measurement to be used concurrently for mapping of the anterior corneal surface. Both of these data sets can, for example, be referenced to the corneal apex determined independently with these measurements themselves, or a CCD camera can be used to determine pupil location during the measurements and the data sets can each be registered to that feature. An embodiment of this invention includes any particular referenced feature or features that enable the epithelial and topography measurements to be synced for registration and selection so measurement information can be used concurrently in a determination of an ablation pattern.

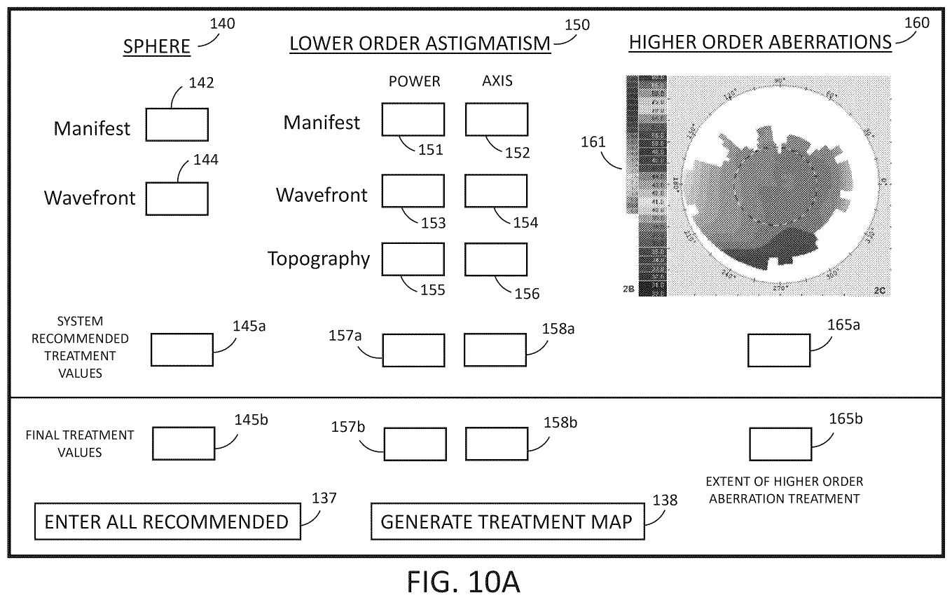

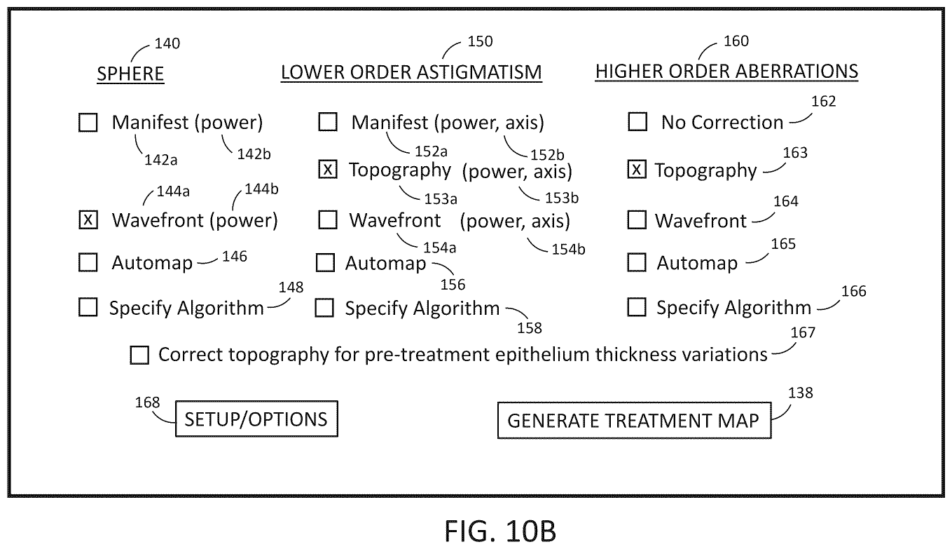

As described in more detail below, a system with the combination of data gathering technologies of FIG. 8 can provide a variety of treatment options for the surgeon. In conjunction with the data visualizations on the display 74, embodiments may include display of measurement modality results and a selection and/or entry of treatment options that can be provided on the user interface. One possible embodiment of such a user-interface is shown in FIG. 10A. Another is shown in FIG. 10B. The treatment map generation software may further comprise thresholds, other processing parameters, and a knowledge base 84 with content and use described further below. Using this information, and on automated analysis of the data from the measurement technologies, suggestions regarding the data on which to base the treatment map can be provided.

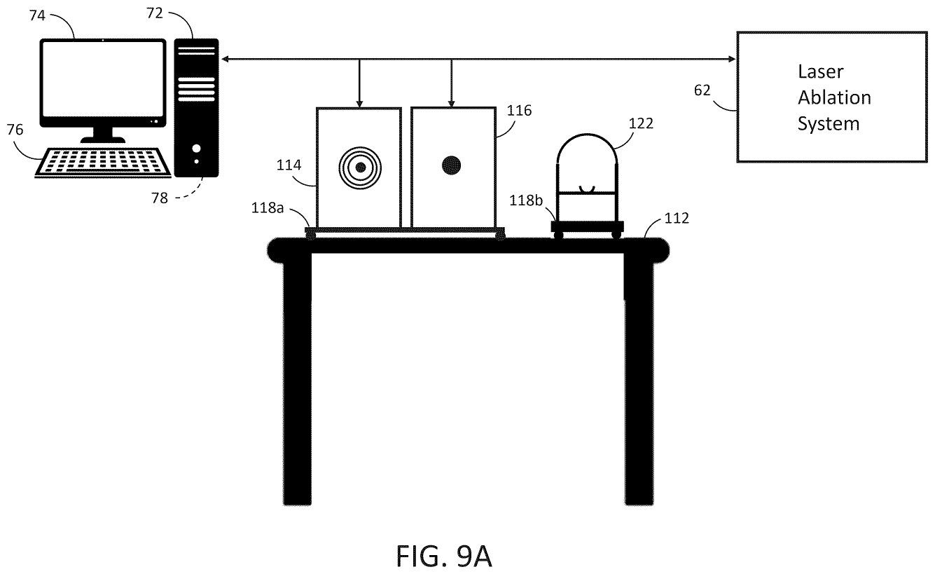

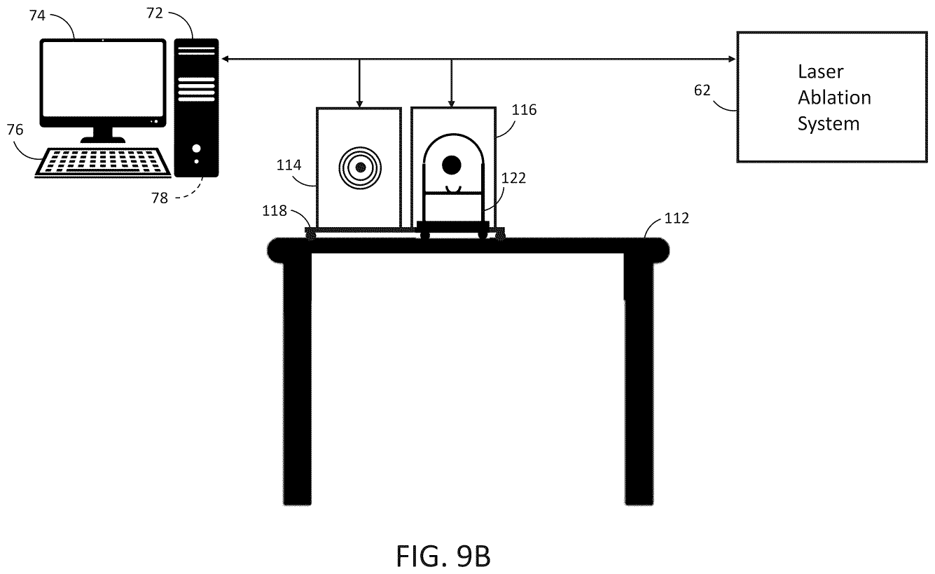

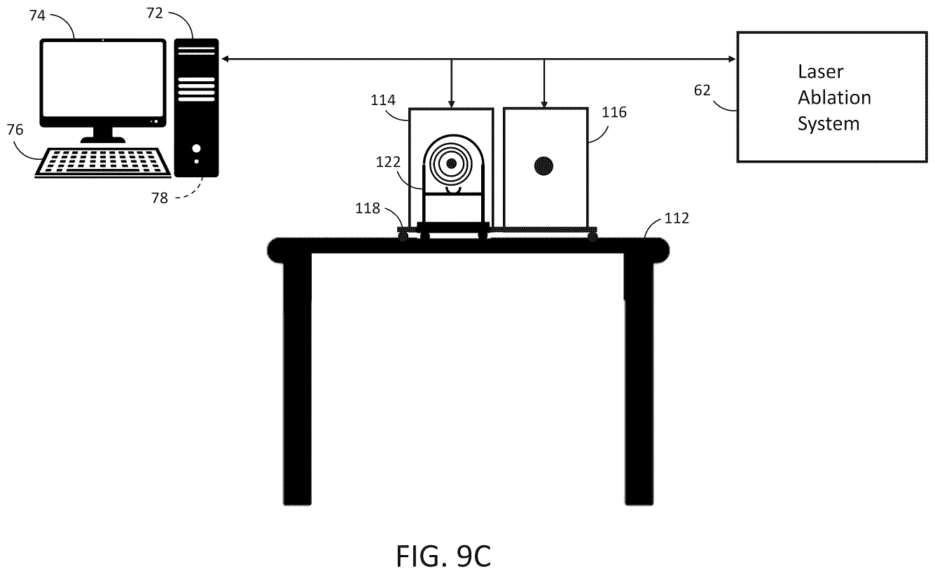

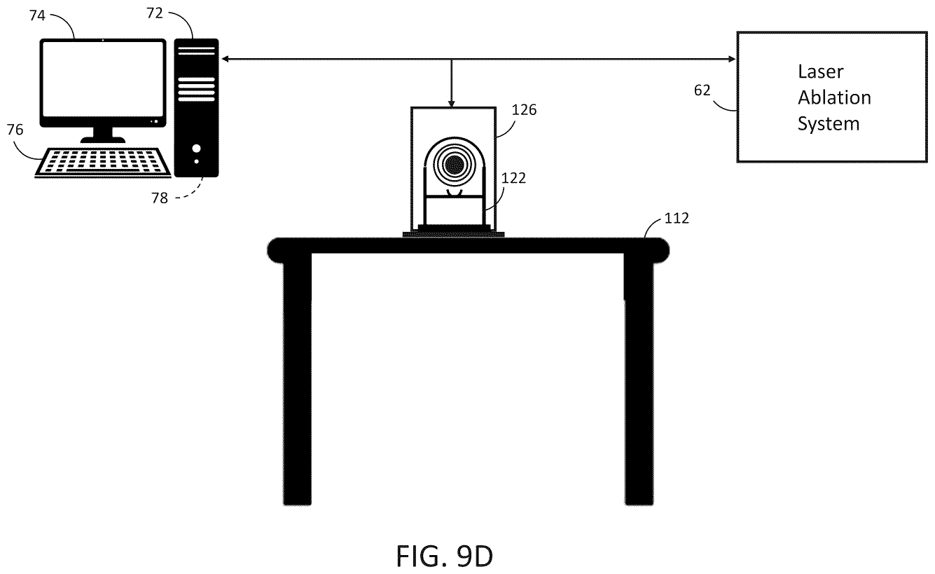

FIGS. 9A to 9C illustrates one exemplary embodiment of a corneal measurement and/or ablation system made in accordance with the principles described above with respect to FIG. 8 that may be in a surgeon's office or clinic for use with a patient. In this embodiment, a platform such as a table, bench, or cart 112 may support one or more measurement instruments 114 and 116 separately housed. A chin rest 122 to support the subject when placing their eyes in front of the instruments may also be provided. Any one or more of the instruments 114, 116 and chin rest 122 may have or be provided on wheels or wheeled carts 118a, 118b or may be otherwise moveable or slidable on the platform 112.

The instrument 114 may be a combined Placido ring corneal topography measuring device combined with a Hartman-Shack wavefront aberrometer. This combines instruments 91 and 92 of FIG. 8 into a single device. A commercially available example of such a device is the Schwind Peramis. Instrument 116 may be an OCT based epithelial mapping instrument such as the Optovue Solix. As shown in FIGS. 9B and 9C, the chin rest 122 and instruments 114 and 116 can be selectively moved in front of and away from the patient to make the measurements in a convenient manner with the subjects head being maintained substantially stationary during the process.

The instruments 114 and 116 are connected for data communication with a data processing system 132 with user display and I/O capabilities 51 such as described above. Also included is the laser ablation system itself 142. The processing algorithms for generating the ablation maps may be all in the processing system 132, all in laser ablation system 142, or distributed between them in any manner.

FIG. 9D illustrates a system similar to that shown in FIGS. 9A through 9C except in this embodiment a single instrument is a combined Placido ring topography measuring device, wavefront aberrometer, and OCT epithelium thickness measuring device.

FIG. 10A is an exemplary user interface that may be provided by software in some implementations of the system of FIG. 8. This user interface may be presented on the display 74 of FIG. 8. In this embodiment, three categories of aberration are presented in three different columns, where each column provides the ability to view different measurement modality results and determine the treatment or aspects thereof to be applied to each category of aberration.

The sphere column 140 may include an output at 142 which may show the outcome of a manifest measurement of the required myopia or hyperopia correction for the subject patient, performed, for example, with a phoropter as described above. This may be entered manually by the user directly into location 142 in this user interface or it may be received by the system through another user interface or another communication method. The sphere column 140 may also include an output at 144 that may show the outcome of a wavefront aberrometry measurement of the required myopia or hyperopia correction for the subject patient, performed, for example, with a Hartmann-Shack wavefront aberrometer as described above. This may be entered manually by the user directly into location 144 in this user interface or it may be received by the system through another user interface or another communication method, such as directly from the aberrometer via a communication channel. The sphere column may further include a location 145a that will show a system recommended correction for myopia or hyperopia that may be incorporated into the treatment map 48d. This value may, for example, be auto-filled by the system with the manifest sphere correction, and may be automatically adjusted to account for the recommended lower order astigmatism correction described below at location 157a.

The lower order astigmatism column 150 may include outputs at 151 and 152 for lower order astigmatism power and axis correction values based on manifest measurements performed, for example, with a phoropter as described above. As with the manifest sphere correction this may be entered manually by the user directly into locations 151 and 152 in this user interface or it may be received by the system through another user interface or another communication method. The lower order astigmatism column 150 may also include outputs at 153 and 154 that may show the outcome of a wavefront aberrometry measurement of the required astigmatism power and axis correction for the subject patient, performed, for example, with a Hartmann-Shack wavefront aberrometer as described above. This may be entered manually by the user directly into locations 153 and 154 in this user interface or it may be received by the system through another user interface or another communication method, such as directly from the aberrometer via a communication channel. The lower order astigmatism column 150 may also include outputs at 155 and 156 that may show the outcome of a corneal topography measurement of the required astigmatism power and axis correction to form a spherical anterior corneal surface, performed, for example, with a Placido ring topographic measurement device as described above. This may also be entered manually by the user directly into locations 155 and 156 in this user interface or it may be received by the system through another user interface or another communication method, such as directly from the topography measurement device via a communication channel. The lower order astigmatism column 150 may further include locations 157a and 158a that will show a system recommended correction for lower order astigmatism that may be incorporated into the treatment map 48d.

Determining an appropriate correction for lower order astigmatism to be used in the treatment map can be difficult, especially because the power and axis correction values determined by the different measurement modalities manifest, wavefront, and topography can be significantly different. Accordingly, advantageous embodiments of the system will auto-fill the locations 157a and 158a with a system recommended lower order astigmatism treatment. The Inventor's prior studies have shown that when the manifest astigmatism differs from the topography astigmatism value, better visual outcomes are usually obtained when the topography astigmatism value is used for the treatment map. A different issue arises however if the topography astigmatism value and the wavefront astigmatism value differ significantly. This situation often indicates that astigmatism is arising from posterior sources such as the lens. In this situation, it may be advantageous to use all or part of the wavefront astigmatism measurement 153, 154 for generating the treatment map.

To auto-fill locations 157a and 158a with a system recommended lower order astigmatism treatment, the system may use a predefined rule for selecting between topography and wavefront or creating a derivative of the topography and wavefront measurements for generating the astigmatism correction aspect of the treatment map. Such a rule may use the thresholds 84 shown in FIG. 8, comparing one or more differences between the topography astigmatism measurements 155 and 156 and the wavefront astigmatism measurements 153 and 154 to the thresholds to select between the topography measurements for astigmatism at 155 and 156 or the wavefront measurements for astigmatism at 153 and 154 to utilize when auto-filling locations 157a and 158a. The threshold for astigmatic power may, for example, be between plus or minus 0.1 and 0.75 diopters, and the threshold for astigmatic axis may, for example, be between plus or minus 1 and 15 degrees. The thresholds 84 may be predefined in the system, and the thresholds may be user settable through a part of the user interface 82. The rule used when the system auto-fills locations 157a and 158a may be to compare the topography power and axis 155, 156 with the wavefront power and axis 153, 154 and utilize the wavefront power and axis 153, 154 for the 157a and 158a entries if the difference for one or both the power and axis is greater than the respective threshold, and otherwise, use the topography measured power and axis 155 and 156 for the 157a and 158a entries. An alternative rule may, for example, be to use a weighted average of the topography correction 155, 156 and the wavefront correction 153, 154 for the entries at locations 157a and 158a. The weights may be equal, resulting in an astigmatism correction that is the average of the topographic correction 155, 156 and wavefront correction 153, 154. In other embodiments, the weights may be a function of the differences between the topographic astigmatism measurements 155, 156 and the wavefront astigmatism measurements 153, 154.

The system may additionally or alternatively use a machine learning algorithm to perform the auto-fill function for locations 157a and 158a. The system may use the knowledge base 84 of FIG. 8 to generate and/or update the rule used to perform the auto-fill function. In some embodiments, the system can employ a machine learning algorithm that takes as inputs one or more of the topographic, wavefront, manifest, OCT, and interferometer measurements as well as possibly other information about the patient such as age, gender, medical conditions, and the like. The machine learning algorithm could be trained to generate a recommended astigmatism treatment for auto-filling locations 157a and 158a based on these inputs using results obtained from prior patients in addition to the wavefront and topography measurements. Such a machine learning algorithm could be updated in real time by the knowledge base 84 as additional patients are treated and results for those procedures are added to the knowledge base.

The higher order aberrations are treated in this example system based on the corneal topography data. Corneal topography data is advantageous because it is high resolution and use of this data for treating higher order aberrations generally leads to smoother more spherical corneal surfaces following surgery which as described above the inventor has found to be especially advantageous. The higher order aberrations column 160 may provide a display of a corneal elevation map 161. The higher order aberrations column 160 may also provide a location 165a with a system recommended value for how many orders of correction depth the treatment map should be designed to treat. This entry 165a may be auto-filled with a default value such as 3, which may specify a treatment map with corrections for trefoil and coma higher order aberrations. Although corneal topography is believed at this time to be the superior choice for treating higher order aberrations with this system, alternative systems using wavefront data for higher order aberrations are also possible.

The user interface may also include a set of corrections that will be incorporated into the treatment map 48d. A selection button 137 may be provided that populates the sphere correction 145b, lower order astigmatism correction 157b, 158b, and higher order correction depth 165b with the system recommended values. If desired by the surgeon, one or more of these system recommended values may be overridden by the user manually entering different values. For example, based on the user's review of the data in locations 142 and 144 the user may enter an average or other derivative of the information provided in the locations 142 and 144 into location 145b. Similarly, the surgeon may be allowed to override the recommended lower order astigmatism treatment recommended by the system by, for example, clicking on locations 157b and 158b and entering different values as determined by the judgment of the surgeon. If desired, the user may enter a different value such as 4 or 5 in location 165b to correct additional or fewer higher order aberrations. After the values for the treatment map at locations 145b, 157b, 158b, and 165b are populated to the satisfaction of the surgeon, a generate treatment map button 138 may be selected to generate the treatment map in accordance with the populated values 145b, 157b, 158b, and 165b.

FIG. 10B is an exemplary menu style user interface that may be used in some implementations of the system of FIG. 8. In this implementation, the user is given nearly complete control of how the different measurement modalities contribute to the final treatment map. As with the user interface of FIG. 10A, this user interface may be presented on the display 74 of FIG. 8. It will be appreciated that a given system for corneal laser ablation may provide only a subset of the user options provided in FIG. 10B. As shown in FIG. 10B, a collection of check box choices 130 for laser ablation treatment map 48d creation may be presented to the physician or other user. In this embodiment, as was the case with the embodiment of FIG. 10A, three categories of aberration are presented in three different columns, where each column provides the ability to select the treatment to be applied to each category of aberration.

The sphere column 140 allows as two of the options a correction for myopia or hyperopia with a manifest measurement 142a or a wavefront measurement 144a. To assist in making a decision, the sphere correction power that will be applied from manifest measurement 142b and the sphere correction power 144b that will be applied from wavefront measurement can be presented to the user on the same display. Another option for sphere correction may be provided by an “automap” option 146. This option may cause the system to select the sphere correction for the treatment map in an automated matter. The system selection may correspond to a predefined rule for selecting between manifest and wavefront or deriving a correction from the manifest and wavefront measurements. The automap function may use the knowledge base 84 of FIG. 8 to generate and/or update the rule used to perform the automap 146 sphere correction determination. In some embodiments, the automap function can employ a machine learning algorithm that takes as inputs one or more of the topographic, wavefront, manifest, OCT, and interferometer measurements as well as possibly other information about the patient such as age, gender, medical conditions, and the like. The machine learning algorithm could be trained to generate a treatment map based on these inputs using results obtained from prior patients. Such a machine learning algorithm could be updated in real time by the knowledge base 84 as additional patients are treated and results for those procedures are added to the knowledge base. More flexibility may be provided by a “specify algorithm” option 148. If this box is selected, the user may be taken to a different screen which allows the user to select from a set of sphere correction algorithm options to choose from. A predefined selectable algorithm may, for example, be the average of the manifest and wavefront corrections. The system may also allow the user to create alternative algorithms for their own use that once defined by the user and entered into the system may be selected for sphere correction via the “specify algorithm” checkbox 146.

The lower order astigmatism column 150 may allow as three of the options a correction for astigmatism with a manifest measurement 152a, a topography measurement 153a, or a wavefront measurement 154a. To assist in making a decision, the astigmatism correction power and axis that will be applied from manifest measurement 142b, topography measurement 153b, and wavefront measurement 154b can be presented to the user on the same display. In general, these three astigmatism values will differ from each other. The Inventor's prior studies have shown that when the manifest astigmatism differs from the topography measured astigmatism value, better visual outcomes are usually obtained when the topography measured astigmatism value is used for the treatment map. A different issue arises however if the topography measured astigmatism value and the wavefront measured astigmatism value differ significantly. This situation often indicates that astigmatism is arising from posterior sources such as the lens. In this situation, it may be advantageous to use the wavefront astigmatism measurement 154a, 154b or some other variant of the wavefront measured astigmatism measurement for generating the treatment map. With the display shown in FIG. 10B, the power and axis values 153b provided for the topography measurement and the power and axis values 154b provided for the wavefront measurement can be compared by the user. If the user concludes from such a comparison that the difference between the topography power and/or axis 153b is different from the wavefront power and/or axis 154b by an amount that is large enough that posterior astigmatism is indicated, the user may select the wavefront measurement 154a for use in generating the treatment map or use a variant of the two measurements. The user may have certain threshold differences in power and/or axis in mind when making the decision about whether to use topography 153a, wavefront 154a, or a variant for the astigmatism correction aspect of the treatment map.

As with the sphere correction, another option for lower order astigmatism correction may be provided by an “automap” option 156. This option may cause the system to select the astigmatism correction for the treatment map. The system selection may correspond to a predefined rule for selecting between topography and wavefront or creating a derivative of the topography and wavefront measurements for generating the astigmatism correction aspect of the treatment map. The automap function may use the thresholds 84 shown in FIG. 8, comparing one or more differences between the topography astigmatism measurement 153a and the wavefront astigmatism measurement 154a to the thresholds to select between the topography measurement for astigmatism 153a or the wavefront measurement for astigmatism 154a when generating the treatment map. The threshold for astigmatic power may, for example, be between plus or minus 0.1 and 0.75 diopters, and the threshold for astigmatic axis may, for example, be between plus or minus 1 and 15 degrees. The thresholds 84 may be predefined in the system, the thresholds may be user settable through a part of the user interface 82. The algorithm used when the automap function 156 is selected for astigmatism may be to compare the topography power and axis 153b with the wavefront power and axis 154b and utilize the wavefront power and axis 154b for the treatment map if the difference for one or both the power and axis is greater than the respective threshold, and otherwise, use the topography measured power and axis 153b. The automap function may also use a machine learning algorithm such as described above with the sphere correction. Also as with the sphere correction, more flexibility may be provided by a “specify algorithm” option 158 for the astigmatism correction. If this box is selected, the user may be taken to a different screen which allows the user to select from a set of astigmatism correction algorithm options to choose from. A predefined selectable algorithm may, for example, be a weighted average of the topography correction 153b and the wavefront correction 154b. The weights may be equal, resulting in an astigmatism correction that is the average of the topographic correction 153b and wavefront correction 154b. In other embodiments, the weights may be a function of the differences between the topographic astigmatism measurement and the wavefront astigmatism measurement. The system may also allow the user to create alternative algorithms for their own use that once defined by the user may be selected for astigmatism correction via the “specify algorithm” checkbox 146.

A column 160 for selecting correction strategies for higher order aberrations is also provided in the example user interface of FIG. 10B. A first option 162 of no correction for higher order aberrations is provided in this embodiment. This may be selected if the surgeon wishes to treat a subject using only traditional manifest data. In such an instance, a surgeon may select manifest 142a for sphere, manifest 152a for lower order astigmatism, and no correction 162 for higher order corrections. Similar to the sphere column 140 and lower order astigmatism column 150, the higher order aberrations option column 160 provides the option to select higher order corrections based on topography measurements at 163 or wavefront measurements 164. Also similar to the sphere correction column 140 and the lower order astigmatism correction column 150 another option for higher order aberrations correction may be provided by an “automap” option 165. This option may cause the system to select the higher order aberration correction for the treatment map in an automated matter. The system selection may correspond to a predefined rule for selecting between topography and wavefront or deriving a correction from the topography and wavefront measurements. The automap function may use the knowledge base 84 of FIG. 8 to generate and/or update the rule used to perform the automap 165 higher order aberration correction determination. The automap function 165 may also use a machine learning algorithm such as described above with the sphere correction. More flexibility may be provided by a “specify algorithm” option 166. If this box is selected, the user may be taken to a different screen which allows the user to select from a set of higher order aberration correction algorithm options to choose from. A predefined selectable algorithm may, for example, allow the surgeon to correct only certain Zernicke aberrations such as only coma, but not correct other higher order aberrations. Other options for specialized algorithms available from this option 166 may be to correct some higher order aberrations based on wavefront measurements and other higher order aberrations based on topography measurements. The system may also allow the user to create alternative algorithms for their own use that once defined by the user may be selected for the higher order aberration correction via the “specify algorithm” checkbox 166.

An option can also be provided in the menu interface of FIG. 10B for epithelial compensation in the treatment for differences in pre-treatment epithelium thickness at selection 167. This selection can incorporate information from the epithelium thickness measuring instrument 93 of FIG. 8. This epithelial compensation can be incorporated into the treatment map in different ways depending on whether a PRK or LASIK procedure is being performed. If PRK is being utilized, laser ablation of the epithelium to a depth of approximately the thickest part of the epithelium in the treatment zone may be performed. This will cause ablation of stromal tissue as well in those areas where the epithelium is thinner than the thickest portion. This will alter the corneal topography because some raised portions will be lowered and these changes can be taken into account in topographically guided corrections for HOA, astigmatism, and defocus that are incorporated into the ablation map. If a LASIK procedure is being performed, a local point by point subtraction of epithelium thickness from the topographically measured height values can be performed; which results in an accurate topographic map of the stromal tissue itself. After lifting the flap, the ablation map can provide ablation in accordance with the epithelium corrected topology. After flap replacement, the stromal surface will be smoother, resulting in a more even epithelium layer after healing. This option 167 can be selected or not in association with any combination of selections from the correction columns 140, 150, or 160 described above.

In some embodiments, the automap and/or specify algorithm options of FIG. 10 may include the option of using ray tracing algorithms for generating the treatment map. Ray tracing has been experimented with for over a decade and it continues to have several significant flaws in the methodology as in its existing use. Problems with a ray tracing methodology that uses wavefront include the following: (1) It continues to follow the scientific postulation that posterior ocular astigmatism is present in a significant number of patients; therefore, requiring “whole” eye measurements and the use of data responsible for suboptimal outcomes. It ignores research by the Inventor that demonstrates that the majority of suboptimal outcomes have nothing to do with posterior ocular astigmatism, but rather other factors, such as, epithelial compensation and LASIK flap induced corneal shape change, (2) It ignores the fact that crystalline lens changes could affect the outcome of the measurements, as the lens changes as a person ages. This could make data in patients over the age of 50 inaccurate and create an incorrect laser profile, (3) It uses conventional and existing understanding of the epithelial layer, which is not considered to be variable in the normal Gullstrand models of the eye; in other words, it uses data that considers the epithelium to be an even thickness blanket over the anterior cornea following the shape of the cornea underneath. As such, existing methodologies do not add or change a wavefront-based map for changes in epithelial thickness.

Embodiments as described herein that provide epithelium thickness information can be used to improve ray-tracing algorithms by taking this thickness information into account when developing the ray tracing eye model for treatment map generation.

Another interface feature illustrated in FIG. 10B is a setup/options button 168. Selecting this button may cause display of one or more user interface screens through which the surgeon can, for example, set the thresholds 84 in FIG. 8 that may be used in the automap function 156 and define algorithms that are available for treatment map generation using the select algorithm options 148, 158, and 166.

After the surgeon makes their selections from columns 140, 150, and 160, they can select the button 169 to initiate the generation of the treatment map in accordance with the selections.

The menu driven treatment map generation interface of FIG. 10B allows a surgeon to generate treatment maps using a wide variety of clinical protocols. As mentioned above, for example, a traditional pure manifest treatment map can be generated by selecting options 142, 152, and 162. A wavefront guided treatment map can be generated by selecting options 144, 154a, and 164. A topographic guided treatment map can be generated by selecting options 142, 153a, and 163. As will be described further below with some specific examples, the system can allow optimal use of the different measurement modalities, especially the topography and wavefront measurement modalities, which provide both overlapping and complementary information regarding the optical characteristics of a subject eye. Depending on the measurements themselves, their quality, and/or their availability, either topographic information alone, wavefront information alone, or a selective combination of topographic and wavefront information can be used based on the surgeon's judgement for each particular patient.

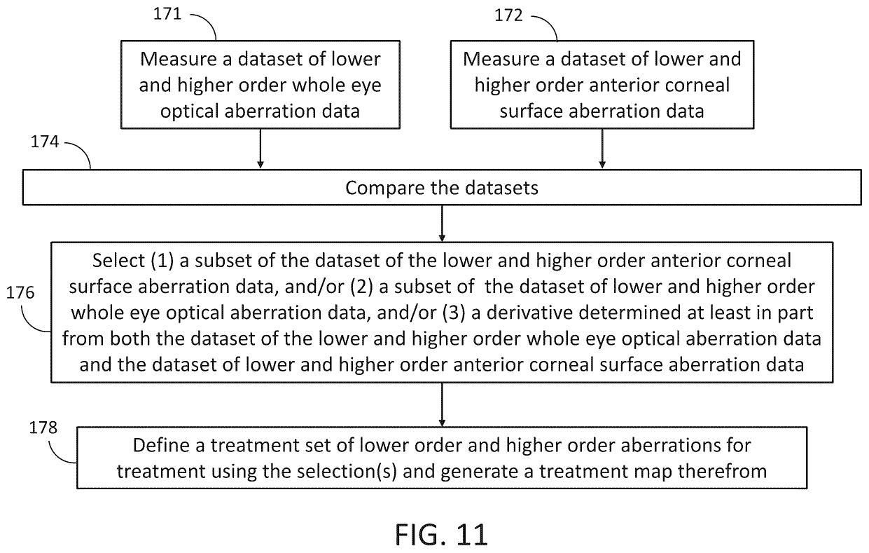

FIG. 11 is a flowchart that illustrates a method of selection of one or both of topographic information and wavefront information by a surgeon or other user of the system that may be enabled and facilitated by the systems of FIGS. 8-10B described above. Referring now to this FIG. 11, at block 171 a dataset of lower order and higher order whole eye optical aberration data is measured. At block 172 a dataset of lower order and higher order anterior corneal surface aberration data is measured. At block 174, the two datasets are compared. Based at least in part on the comparison performed at block 174, a selection from multiple options is performed. As shown in block 176, a subset (which may be any portion of or all of the dataset of block 171) of the whole eye optical aberration data may be selected, and/or a subset (which may be any portion of or all of the dataset of block 172) of the anterior corneal surface aberration data may be selected, and/or a derivative determined at least in part from both the whole eye optical aberration data and anterior corneal surface aberration data may be selected. At block 178, based at least in part on the selection(s) made, a treatment set of lower order and higher order aberrations for correction are defined, and a treatment map is generated therefrom.

In some cases, more than one of the options of block 176 can be used for generating a single treatment map. In addition, other factors such as manifest measurements and/or epithelium thickness related measurements can be used as part of generating a treatment map along with the selections of the options in block 176 that are illustrated in FIG. 11. For example, a surgeon may, after comparing the datasets at block 174, decide to follow the first option of block 176 and use topographic measurements for lower order astigmatism and higher order aberrations and use manifest measurements for sphere. In this case, the second and third options of block 176 are not used, and the wavefront measurements of block 171 may be ignored for the generation of the treatment map. For another patient, a surgeon may select both the first and second options of block 176, using, for example, wavefront measurements for sphere and topographic measurements for lower order astigmatism and higher order aberrations. Another example of using both the first and second options of block 176 for generating a single treatment map is using the wavefront measurements for both sphere and lower order astigmatism and using topographic measurements for higher order aberrations.

The third option of block 176 comprises selecting a derivative determined at least in part from both the dataset of lower and higher order whole eye optical aberration data and the dataset of lower and higher order anterior corneal surface aberration data. Such a derivative can take a variety of forms and be generated in a variety of ways. Different algorithms for computing such derivatives can be available to the surgeon through the specify algorithm options 148, 158, and 166 of FIG. 10 for example. In one example derivative computation, the lower order astigmatism component of the whole eye optical aberration data and the lower order astigmatism component of the anterior corneal surface aberration data can be combined in selected percentages as a weighted average. If the weights are selected equal, then 50% of the correction indicated by the whole eye lower order astigmatism is added to 50% of the correction indicated by the anterior corneal surface aberration data. As another alternative to this scenario, the weights could be adjusted depending on the difference between the whole eye astigmatism aberration correction and the anterior corneal surface aberration astigmatism correction. For example, a large difference between whole eye astigmatism and corneal surface astigmatism may indicate that astigmatism is arising from posterior sources such as the lens. To take this into account, an algorithm may increase the weight of the whole eye aberration astigmatism relative to the anterior corneal surface astigmatism as the difference between the whole eye astigmatism and the anterior corneal surface astigmatism increases. As described above, another alternative algorithm may weight the anterior corneal surface aberration astigmatism at 100% and the whole eye astigmatism aberration at 0% unless the difference between the whole eye aberration astigmatism and the corneal surface aberration astigmatism is greater than a threshold, in which case the anterior corneal surface aberration astigmatism is weighted at 0% and the whole eye aberration astigmatism is weighted at 100%. This type of algorithm can be conceptualized as using the anterior corneal surface aberration data astigmatism as a default starting point, and then modifying or replacing that astigmatism value with the whole eye aberration astigmatism data under appropriate conditions that may be pre-defined or evaluated on-the-fly by the surgeon. A derivative may also be generated from a machine learning algorithm such as described above. All of the computations, selections, derivatives, and the like described herein applied to lower order astigmatism could also be applied to higher order aberrations such as coma and trefoil.

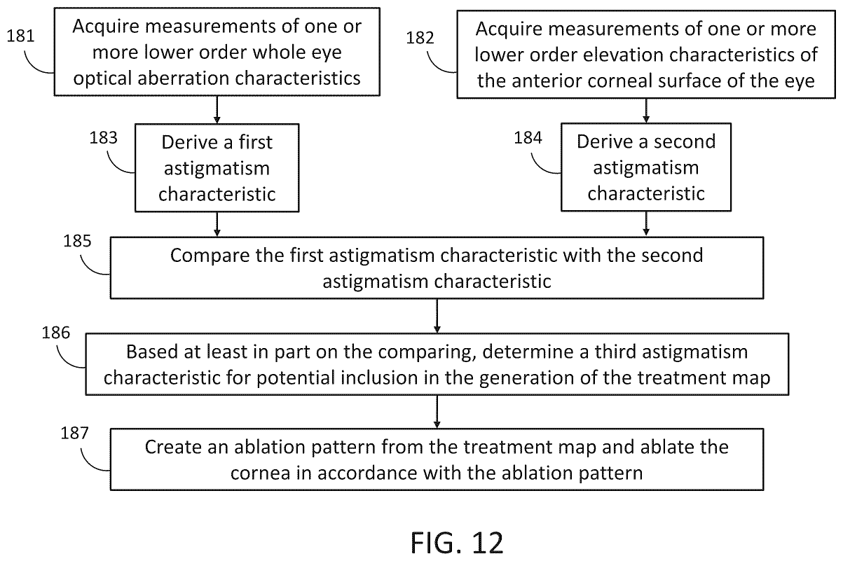

FIG. 12 is an illustration of one method of treating astigmatism according to some embodiments. The method of FIG. 12 is another example of a treatment protocol or portion thereof that may be performed in accordance with the several options set forth in block 176 of FIG. 11 and is especially applicable to the embodiment shown in FIG. 10A. Referring now to FIG. 12, at block 181 measurements of one or more lower order whole eye optical aberration characteristics are acquired, and at block 183 a first astigmatism characteristic is derived therefrom. This first astigmatism characteristic may correspond to the entries in locations 153 and 154 of FIG. 10A. At block 182, measurements of one or more lower order anterior corneal surface elevation characteristics are acquired, and at block 184 a second astigmatism characteristic is derived therefrom. This second astigmatism characteristic may correspond to the entries in locations 155 and 156 of FIG. 10A. At block 185, the first and second astigmatism characteristics are compared. At block 186, based at least in part on the comparing, a third astigmatism characteristic is determined for potential inclusion in the generation of a treatment map. This third astigmatism characteristic may correspond to the entries in locations 157a and 158a and/or the entries in locations 157b and 158b of FIG. 10A. At block 187, an ablation pattern is created from the treatment map, and the subject cornea is ablated in accordance with the ablation pattern. As described above, the third astigmatism characteristic can be generated from the first and second astigmatism characteristics in a variety of ways.

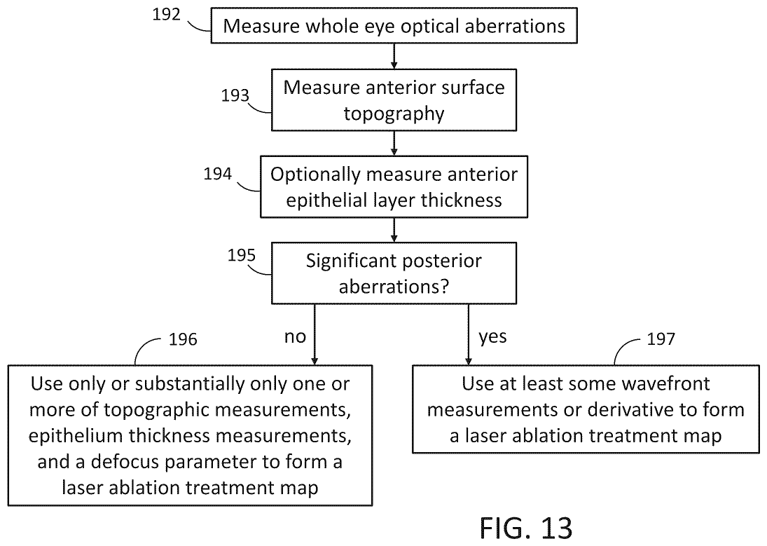

Referring now to FIG. 13, another exemplary method is illustrated. The method of FIG. 13 is another example of a treatment protocol or portion thereof that may be performed in accordance with the several options set forth in block 176 of FIG. 11. In this method, at block 192, whole eye optical aberrations are measured. This may be performed with the wavefront aberrometer 92 of FIG. 8. At block 193, anterior surface topography of the eye is measured, such as with the topology measuring instrument 91 of FIG. 8. At block 194, epithelium thickness is optionally measured, such as with the epithelium thickness measuring instrument 93 of FIG. 8.

At block 195, a determination is made regarding the distribution of the aberrations of the eye between characteristics of the anterior ocular portion and the posterior ocular portion. This determination may be more specifically directed to the question of whether characteristics of the posterior ocular region are a significant cause of the measured optical aberrations for the whole eye which can be measured with, for example, the wavefront aberrometer. By comparing the whole eye aberration data gathered at block 192 with the anterior surface topography data of block 193, information regarding the separate contribution of anterior and posterior ocular structures to the detected refractive aberrations for the whole eye can be determined. For example, both the wavefront aberrometer data and topography data may be processed to generate independent astigmatism correction values to be performed with the laser ablation. As shown in FIG. 13, if these values are similar, it can be concluded at block 195 that posterior structures do not contribute significantly to the astigmatic aberrations of the eye. In this case, it can be advantageous to follow the “no” path of FIG. 13 to block 196 and to use only or substantially only one or more of topographic measurements, epithelium thickness measurements, and a defocus parameter to form a laser ablation treatment map. This pathway performs laser ablation in a manner that will form a smooth, close to spherical contour for the anterior corneal surface, without attempting to create an imperfect anterior corneal surface that compensates for theoretical posterior sources of optical aberration. If, however, the whole eye astigmatism measured with the wavefront aberrometer differs substantially from the astigmatism present on the anterior surface of the eye as measured topographically then it may be concluded that there could be astigmatic aspects to posterior ocular structures such as the posterior corneal surface and/or the lens. In this case, the method can follow the “yes” path to block 197, where at least some wavefront aberrometer measurements or derivative are used to form a treatment map.

Following the “yes” path may involve using wavefront aberration measurements for any one or more than one aberration correction. In some situations, only wavefront astigmatism may be used as part of the treatment map and the rest of the corrections could be based on topography and manifest measurements. In some situations, data from the wavefront aberrometer 92 may be the primary or even sole source of data used to generate a treatment map 48d.

### Lenticular Astigmatism Measurement—Cataract Eye Surgeries

It may be noted that the systems and methods described herein can be used to determine a specific value for lens astigmatism. Posterior ocular lower order astigmatism and higher order aberrations (those posterior to the anterior corneal surface) has four main components when the light path is followed. These are the posterior corneal surface which can be measured by Scheimpflug imaging which has been shown to cause small amounts of astigmatism, the lens which cannot be imaged or measured directly but is likely responsible for those patients with significant posterior ocular astigmatism, the vitreous, which may add aberrations but not astigmatism through syneresis but this cannot be measured, and the retina, which may cause small amount of astigmatism which is measurable by OCT imaging. If posterior ocular astigmatism is defined as any astigmatism posterior to the anterior cornea, this leaves posterior corneal astigmatism, lenticular astigmatism, and retinal astigmatism as sources. With the multi-imaging format systems and methods described above, if a patient has wavefront measured astigmatism which is different than the topography measured astigmatism, then the difference is likely due at least in part to posterior ocular astigmatism. If posterior corneal astigmatism is then measured with Scheimpflug, and retinal astigmatism with OCT, a value for lenticular astigmatism can be calculated. This may be very useful for cataract surgeries. Cataract surgeries involve replacing a patient's lens with a replacement lens implant. If the lens being removed has a known amount of astigmatism, reproducing the patient's pre-surgical vision quality after surgery would require the implant to have a similar astigmatism as part of its design. In some cases of cataract surgery, the new lens implant is designed to correct the pre-surgical eye myopia, hyperopia, and astigmatism. When this is being done, knowing how much astigmatism is being removed by taking out the natural lens can be used to better design the implant to correct the remaining sources of astigmatism.

### Glasses, Contacts, and Lens Implants

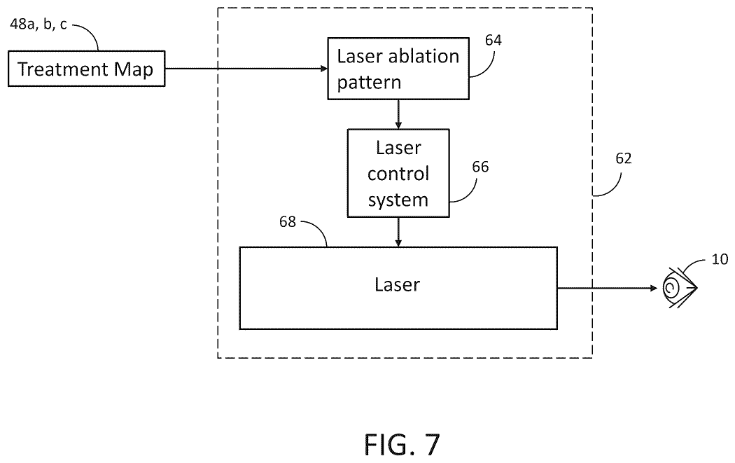

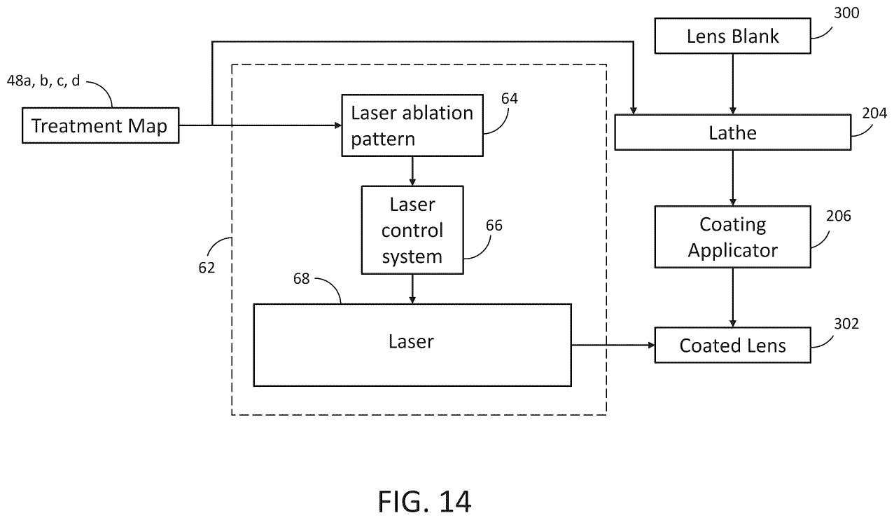

FIG. 14 shows another use for the systems and methods described herein. The system of FIG. 14 is similar to that shown in FIG. 7, where the treatment map 48a, b, c, d, however it is generated, may be used as an input to a laser system 62. A laser ablation pattern 64 is used by a laser control system 66 to control laser position, pulse duration, and the like for the laser 68 to emit the appropriate laser pulses in accordance with the treatment map 48a, b, c, d. In the system of FIG. 14, however, instead of performing ablation on a cornea, the ablation is performed to customize the lens to correct higher order aberrations of the wearer's eyes. As shown in FIG. 14, in one example of this process, a lens blank 300 may be processed by a lathe 204 to generate the desired corrections for lower order aberrations myopia, hyperopia, and lower order astigmatism using a treatment map that can generated by methods and/or systems of this invention. If HOAs are also to be treated, the modified lens can then processed by a coating applicator 206 which affixes a coating layer. The coating layer may be thin, for example less than one millimeter thick and may be designed to not affect the optical performance of the lens as it exists prior to application of the coating layer. The coating layer of the coated lens 302 may then be laser processed with the laser 68 in accordance with the treatment map 48a, b, c, d to imprint a mirror image of the subject's higher order aberrations into the coating layer. The laser may etch the layer material with the higher order aberration corrections. Another alternative is to use a layer material that is heat modifiable, where the imprinting of the higher order aberrations is done by selective heating and local deformation of the coating layer. In another example, a specialized lens with material capable of being directly imprinted may be used. In addition to lenses for glasses, contact lenses and toric lenses for lens replacements in cataract surgery could be processed the same way to incorporate corrections for higher order aberrations into these products as well. Thus, with the system of FIG. 14, glasses, contact lenses, and lens implants for cataract surgeries can be manufactured from the treatment maps generated with the above-described systems and methods which include custom corrections for higher order aberrations.

### Presbyopia Treatment

The systems and methods described above can also be used to execute a novel laser ablation treatment for presbyopia. Presbyopia refers to the loss of near vision due from age related stiffening of the natural crystalline lens. This loss of flexibility prevents the muscles around the lens to flex the lens to change the focus for near work such as reading. As this lens system becomes stiff, it reaches a critical point in the early 40's and progresses where there is virtually no lens flexibility by a person's late 50's or early 60's. This is a problem every human being will contend with, unless they are naturally slightly nearsighted (which obviously compromises their distance vision. Thus, treatments for presbyopia are in high demand, and the search for an effective treatment is ongoing. Currently there are the following commercialized treatments:

Spectacles: reading glasses, bifocal glasses (these have an upper distance correction and a lower fixed reading correction, progressive glasses (these replace the lower fixed reading correction with a variable correction that increases in power as you move your gaze downwards. This has the advantage of added flexibility, but the disadvantage of a smaller working zone for a particular power. Many patients have problems with their vision due to the two or more zones in their glasses, especially with looking down as they are walking, missing steps, curbs etc, and many patients complain they make them dizzy and uncomfortable. Those patients will require completely separate glasses for distance and reading, and switch the two as necessary.

Monovision contact lenses: this technique corrects the dominant eye for distance vision, and the non-dominant eye for close or mid vision. There are limitations on how strong the reading vision can be to allow for neuro-adaptation so the brain can fuse the images and utilize the system, limitations with night glare and night vision because of the different eyes, and also limitations with patients who do not tolerate contacts or have corrections such as high astigmatism that soft contact lenses do not treat well.

Bifocal contact lenses: contacts with two or more circular zones with different powers. See multifocal intraocular lenses below.

Monovision laser vision correction: the same as above, except the correction is done on the cornea. This is better tolerated than contact lenses, as there is no problem with contact lens tolerance. It also creates a stable difference between the two eyes that the brain can neuro-adapt to, and also astigmatism correction is much better via laser than with contacts.

Corneal implants to induce corneal change or extended depth of focus (respectively Raindrop and KAMRA). Complications from corneal response to these as well as placement issues limited their effectiveness, and the side effects of the corneal reaction caused surgeons to stop using them.

Replacement of the natural crystalline lens (during cataract surgery) with an artificial lens with multiple circular focal zones or a lens with a progressive change to the power (similar to a progressive spectacle) allowing for reading power without causing the diffraction problems with multiple zones (Extended depth of focus or EDOF lens). An example is the Vivity intraocular lens by Alcon. These presbyopic treating IOLs have problems as well, as multifocal lenses will diffract light between the zones and patients can have significant night glare and visual issues. The EDOF lenses have the issue of not working if the pupil is not in the right position and the brain does not neuro-adapt to use these extended depth of focus zones.

Some other presbyopic corneal treatments have tried to make a centrally steeper cornea for monovision with some progressive affect but those treatments failed because the corneal steepening affect would decrease over time.

In summation of past treatments, they have either been corneal based (excimer laser, contact lens, and corneal implants), or intraocular lens based (replacing the natural crystalline lens usually when cataract surgery is needed), and they follow the same principles of either multiple zones of refraction, or an extended depth of focus strategy.

Attempts to find corneal refractive laser treatments for presbyopia that utilize either multifocal or EDOF strategies have met with limited success because of some of the same side effects as lenses, but also because the epithelium will compensate for a cornea that does not have a smooth, progressive curvature. The epithelium has been shown to compensate for irregularities in the cornea, and therefore the most successful excimer laser procedures have been with lasers that create smooth laser profiles that minimize epithelial compensation. Furthermore, epithelial compensation can also occur due to the natural higher order aberrations of the cornea. In treating only lower order aberrations such as lower order astigmatism and sphere (as in most LASIK), the change to the natural HOA could cause epithelial compensation that would also affect such corrections as a multifocal cornea or an extended depth of focus cornea.

Some of the failure or deficiencies in prior presbyopic laser correction treatments stems from the same problem that exists in overall laser correction treatments: the focus for treatments as been based on the existing medical and scientific basis that require an ablation pattern derived from “whole” eye measurements, rather than the Inventor's clinical studies that show that an ablation pattern should be based from other data necessary to create as uniform of cornea as possible, not a cornea that is ablated for theoretical posterior irregularities or ignores epithelial compensation effects on HOAs. The novel features of this invention begin with the Inventor's new scientific understanding that any procedure must create as uniform of cornea as possible. Therefore, the base cornea where a presbyopic laser correction treatment can be performed on would ideally be a cornea without significant higher order aberrations that could not only cause distortions in important areas of the extended depth of focus zone (EDOF zone) but could also induce epithelial compensation that would impact this EDOF zone. Therefore, the cornea must be as uniform as possible, which may be accomplished using the 032 patent systems and methodologies utilizing the LYRA™ Protocol to make a uniform cornea. Performing such a procedure on the cornea has advantages over an intraocular lens, as it would be performed on the first, anterior and therefore most important refracting surface. It would also be anterior to the iris, which can block intraocular peripheral EDOF zones.

In the process of making a more uniform cornea, the peripheral shape could be smoothly changed to create an EDOF circular zone to act in a similar way to a progressive spectacle lens which has a change in curvature to change the power to induce more myopia outwardly from the center 3 mm of the laser ablation pattern. This progressive change in curvature may be similar to that found in EDOF intraocular lenses. This curvature change is called the Q ratio, which defines the rate of curvature change across the human cornea. The natural cornea is not a dome, but aspheric. The average change of curvature from periphery to the center of a natural human cornea has a q ratio of 0.28. By manipulating the Q ratio and changing it across the peripheral cornea in an already uniform cornea, an extended depth of focus could be achieved that would not induce epithelial compensation and also would not scatter light with abrupt transition zones that would cause glare and night halos. This induced spherical aberration may cause some haloing around lights, but it likely would be a minimal trade-off for the enhanced reading as spherical aberration is well tolerated and the least disruptive to vision.

Embodiments of this procedure may comprise the following:
The creation of a uniform cornea, which has removed all HOA and corrected lower order astigmatism and sphere, and ablated a central region of the anterior portion of the cornea in accordance with a first Q-factor and ablated a peripheral region around the central region of the anterior portion of the cornea in accordance with a second, different Q-factor. This would be created from a map utilizing the following:
A. Topography measured HOA and lower order astigmatism and axis;
B. Wavefront and/or manifest measured refraction and total ocular astigmatism;
C. A software component that will compare steps A and B to determine if posterior ocular astigmatism exists and to treat accordingly;
D. OCT measured epithelial thickness mapping to provide a map of epithelial compensation of anterior stromal irregularity;
E. A software component that combines the resulting map from A, B, and C together with D to create a final comprehensive map of the cornea.
E. Measurement of the Q ratio from the corneal topography or other device that measures corneal curvature.
F. An ablation pattern that follows the pre-procedure natural q ratio for the central ablation area, likely to be 3 mm, but may be as small as 2 mm or even 1.5 mm.
G. An ablation pattern that then deviates from the central area to the periphery of the ocular ablation zone to provide extended depth of focus (EDOF), usually out to 6-7 mm. This may be referred to as the EDOF zone and may manipulate the Q ratio in a way similar to the defocus curves of EDOF intraocular lenses to allow for reading vision from the extended depth of focus.

A final part of the ablation zone may be from the outer edge of the EDOF zone to about 9 mm, or the transition zone that follows the final original transition zone based on the natural Q ratio or some other Q ratio that will be determined during testing. This is relatively unimportant as the vast majority of patients that are presbyopic have pupils smaller than 6.5 mm.

In summary, this invention treats presbyopia with an enhanced corneal laser ablation system and procedure that includes reshaping a cornea so that it is emmetropic (no correction) in the center region, with the peripheral zone decreasing in power more rapidly to create the myopia zone so that the peripheral zone is the reading area for the subject. Embodiments of this procedure may create a uniform cornea that would be derived from utilizing topography information and/or wavefront and/or scheimpflug camera information combined with OCT epithelial mapping irregularity, but would also change the Q ratio outside the central ablation area (likely 1.5-3 mm) of correction to create a circular EDOF zone that would be similar to a progressive pair of glasses or a EDOF intraocular lens. This new medical procedure by the Inventor is named the Q-RESHAPED™ Protocol (Q-Ratio Enhanced Shape Healing Adjusted Presbyopia Extended Depth Protocol). The system and methods to perform this new breakthrough medical procedure are embodiments of this invention.

The Q-RESHAPED™ Protocol has two fundamental differences from past proposed treatments. First, it focuses on creating a uniform cornea, specifically treating HOAs and epithelium compensation, which past treatments essentially ignored. Second, it treats presbyopia by reshaping a cornea so that it is emmetropic (no correction) in the center region, with the peripheral zone decreasing in power more rapidly to create the myopia zone so that the peripheral zone is the reading area for the subject; whereas, prior treatments attempted to make the central zone the reading area and decrease the induced myopia peripherally-just the opposite of this invention. Prior proposed treatments essentially create an aberrant cornea that will have the epithelium compensate for the aberration and throw the ocular focusing system off.

Epithelium compensation occurs least on a smooth gradual surface. By doing the opposite of prior proposed procedures, we create a smoother gradation, for example using a Q Ratio of 0.8 rather than the natural 0.28. This will prevent epithelial compensation from occurring and filling in the area and ruining the corrective effect.

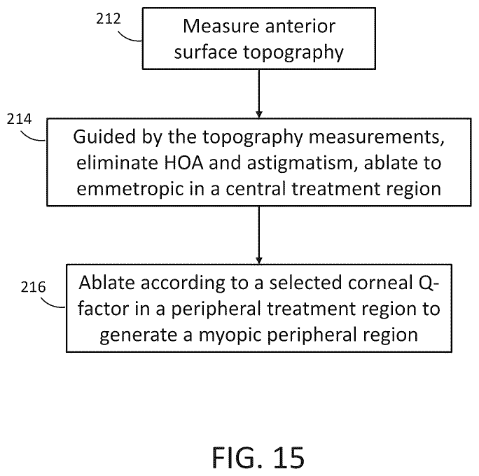

FIG. 15 is a flowchart illustrating the ablation principles described above for treating presbyopia. Referring now to FIG. 15, the method starts at block 212 by measuring the anterior surface topography. With this topographic information generated at 212, ablation is performed to eliminate HOA and astigmatic topographical features of the anterior cornea to produce a smooth substantially emmetropic anterior corneal surface in a central region at block 214. The central emmetropic region to which this topographic guided ablation is applied may be 1.5 to 3 mm in diameter.

At block 216, a peripheral region referred to herein as the extended depth of focus (EDOF) region is created. The EDOF region may extend from the perimeter of the central emmetropic region out to about 6-7 mm diameter. In this EDOF region, Q-factor guided ablation may be used to create a myopic peripheral region. With this ablation treatment for presbyopia, the central region is used for distance vision, and the peripheral myopic region is used for reading.

There are several advantages to this presbyopia treatment method. First, when reading, the visual axis tends to shift downward away from the corneal apex. Because the myopic region used for reading is in the peripheral region rather than the central region, the visual axis will often shift into this myopic region when the subject wants to read. In addition, using topographic guided ablation in the central region and Q-factor guided ablation in the peripheral region helps minimize aberrations at the transition between the central and peripheral regions, which minimizes visual aftereffects created by such aberrations and also minimizes post treatment epithelial compensation that will reduce the myopic curvature introduced by the ablation in the peripheral region.

Although epithelial thickness mapping to provide a map of epithelial compensation of anterior stromal irregularity is considered an important feature of the Q-RESHAPED™ procedure, embodiments of this procedure may include determining epithelial compensation from methodologies other than using an OCT device, such as algorithms to determine theoretical epithelial compensation based on other measuring techniques.

Further, as an alternative to epithelial mapping, embodiments include configuring a wavefront and/or topographic guided ablation system, without OCT, as follows:

Measuring the Q ratio from corneal topography and/or wavefront.

Determining an ablation pattern that follows the pre-procedure natural q ratio for the central ablation area, likely to be 3 mm, but may be as small as 2 mm or even 1.5 mm.

Determining an ablation pattern that then deviates from the central area to the periphery of the ocular ablation zone to provide extended depth of focus (EDOF), usually out to 6-7 mm. This may manipulate the Q ratio in a way similar to the defocus curves of EDOF intraocular lenses to allow for reading vision from the extended depth of focus.

A final part of the ablation zone may be from the outer edge of the EDOF zone to about 9 mm, or the transition zone that follows the final original transition zone based on the natural Q ratio or some other Q ratio that will be determined during testing.

Embodiments of this invention include corneal laser ablation systems configured and programmed to perform the methodologies derived from the disclosures herein. Another option that may be provided in the menu embodiment of FIG. 10B is an option to select a target Q-factor for the anterior cornea following the ablation treatment. With this option, a selected Q-factor can be another guide for ablation map generation.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

1. A method of performing a corneal laser ablation procedure on an eye of a subject, the method comprising:
generating a treatment map from acts comprising:
acquiring measurements of one or more lower order whole eye optical aberration characteristics at least in part via retinal reflection measurements and deriving therefrom a first astigmatism characteristic representative of a lower order whole eye astigmatism;
acquiring measurements of one or more lower order elevation characteristics of the anterior corneal surface of the eye at least in part from one or more of either tear film, anterior epithelium, and/or epithelium basement membrane reflection measurements and deriving therefrom a second astigmatism characteristic representative of a lower order anterior corneal surface astigmatism;
comparing the first astigmatism characteristic with the second astigmatism characteristic to generate an estimate of an amount of difference between lower order whole eye astigmatism and lower order anterior corneal surface astigmatism;
based at least in part on the generated estimate, determining a third astigmatism characteristic for inclusion in the generation of the treatment map, wherein the third astigmatism characteristic is determined using either (1) one or both of the first astigmatism characteristic and the second astigmatism characteristic or (2) a derivative of one or both of the first astigmatism characteristic and the second astigmatism characteristic; and
generating the treatment map using at least in part the third astigmatism characteristic;
creating an ablation pattern that can be used by a corneal laser ablation device from the treatment map; and
using a corneal laser ablation device to apply laser pulses to the eye in accordance with the ablation pattern.

2. The method of claim 1, comprising comparing the generated estimate to a threshold.

3. The method of claim 2, wherein the threshold is user settable.

4. (canceled)

5. (canceled)

6. (canceled)

7. The method of claim 2, wherein the first astigmatism characteristic, second astigmatism characteristic, third astigmatism characteristic, the estimate, and the threshold each comprise both a magnitude value and an axis value.

8. The method of claim 7, wherein the third astigmatism characteristic is equal to or approximately equal to the second astigmatism characteristic when either or both of the estimate magnitude value is less than or equal to the threshold magnitude value and the estimate axis value is less than or equal to the threshold axis value.

9. The method of claim 7, wherein the third astigmatism characteristic is equal to or approximately equal to the first astigmatism characteristic when either or both of the estimate magnitude value is greater than or equal to the threshold magnitude value or the estimate axis value is greater than or equal to the threshold axis value.

10. The method of claim 1, wherein the third astigmatism characteristic is equal to or approximately equal to a derivative determined using one or both the first astigmatism characteristic and the second astigmatism characteristic.

11. The method of claim 1, wherein the third astigmatism characteristic is an output of an algorithm that has one or both of the first astigmatism characteristic and the second astigmatism characteristic as inputs.

12. (canceled)

13. (canceled)

14. (canceled)

15. (canceled)

16. (canceled)

17. (canceled)

18. (canceled)

19. The method of claim 1, wherein comparing the first astigmatism characteristic with the second astigmatism characteristic comprises evaluating astigmatism characteristics of the eye that are posterior to the anterior corneal stromal tissue surface.

20. The method of claim 19, wherein the astigmatism characteristics of the eye that are posterior to the anterior corneal stromal tissue surface comprise one or more of lenticular astigmatism, posterior corneal astigmatism, and retinal astigmatism.

21. (canceled)

22. (canceled)

23. (canceled)

24. (canceled)

25. (canceled)

26. (canceled)

27. (canceled)

28. The method of claim 1, wherein the generation of the treatment map further comprises:
acquiring measurements of one or more higher order elevation characteristics of the anterior corneal epithelium surface of the eye by one or more of either: retinal reflection measurements, tear film reflection measurements, or anterior epithelium reflection measurements;
acquiring measurements over at least a portion of the stromal tissue of the cornea of one or more of either epithelial thickness characteristics, epithelial basement membrane characteristics, or Bowman's layer characteristics;
deriving corneal stromal tissue elevation characteristics by modifying one or more acquired measurements of higher order elevation characteristics of the anterior corneal epithelium surface using one or more of either: the epithelial thickness measurements, the epithelial basement membrane characteristics, or the Bowman's layer characteristics; and
generating the treatment map using at least in part the corneal stromal tissue elevation characteristics and the third astigmatism characteristic.

29. The method of claim 28, wherein the modification of one or more acquired measurements of higher order elevation characteristics is determined by using one or more of either: the epithelial thickness measurements, the epithelial basement membrane characteristics, or the Bowman's layer characteristics at least in part to calculate the elevation characteristics of the anterior corneal stroma that are being affected by epithelium compensation.

30. (canceled)

31. (canceled)

32. (canceled)

33. (canceled)

34. The method of claim 1, wherein a defocus parameter for the eye of the subject is measured and such measurement is used at least in part to generate the treatment map.

35. (canceled)

36. A method of generating a treatment map in order to create an ablation pattern that can be used for performing a corneal laser ablation procedure on an eye, the method comprising:
measuring a dataset of lower and higher order whole eye optical aberration data;
measuring a corresponding dataset of lower and higher order anterior corneal surface aberration data;
comparing the dataset of anterior corneal surface aberration data with the corresponding dataset of whole eye optical aberration data;
based at least in part on the comparing, performing one or more of (a) through (f):
(a) selecting a subset of the measured corneal surface aberration dataset for a corresponding subset of lower order and/or higher order aberration treatment;
(b) selecting a subset of the measured whole eye optical aberration dataset for a corresponding subset of lower order and/or higher order aberration treatment;
(c) selecting a derivative determined at least in part from both the measured whole eye optical aberration dataset and the measured corneal surface aberration dataset for a subset of lower order and/or higher order aberration treatment;
(d) selecting a derivative determined from an algorithm using the selected subset of corneal surface aberration data and a corresponding selected subset of whole eye optical aberration data as inputs for a subset of lower order and/or higher order aberration treatment;
(e) selecting a derivative determined by an output of a computation that uses a first percentage of a subset of the measured corneal surface aberration dataset and a second percentage of a corresponding subset of the measured whole eye optical aberration dataset as inputs for a subset of lower order and/or higher order aberration treatment;
(f) selecting a derivative determined from an algorithm using the difference in a selected measured subset of whole eye optical aberration data and a selected measured subset of corneal surface aberration data as inputs for a subset of lower order and/or higher order aberration treatment;
using one or more of the chosen options to define a treatment set of lower order and/or higher order aberrations for correction; and
combining the treatment set of lower order and/or higher order aberrations for correction to generate the treatment map.

37. The method of claim 36, comprising replacing and/or modifying measured corneal surface lower order astigmatism using at least in part measured whole eye lower order astigmatism to generate the treatment map.

38. The method of claim 37, comprising replacing and/or modifying at least one higher order corneal surface aberration using at least in part at least one corresponding measured higher order whole eye aberration.

39. (canceled)

40. (canceled)

41. (canceled)

42. (canceled)

43. The method of claim 36, wherein the comparing of the datasets of whole eye optical aberration data and anterior corneal surface aberration data comprises evaluating astigmatism characteristics of the eye that are posterior to the anterior corneal stromal tissue surface.

44. (canceled)

45. The method of claim 36, comprising generating an estimate of an amount of difference between the dataset of whole eye astigmatism and the dataset of anterior corneal surface astigmatism and comparing the estimate to one or more thresholds.

46. The method of claim 45, wherein if the differences between the dataset of anterior corneal surface aberration data and the dataset of whole eye optical aberration data is less than one or more threshold values, the measured anterior corneal surface aberration data is used to generate the treatment map.

47. The method of claim 45, wherein if the differences between the dataset of anterior corneal surface aberration data and the dataset of whole eye optical aberration data are greater than the one or more threshold values, a combination of corneal surface aberration data and whole eye optical aberration data is used to generate the treatment map.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)
